(19) United States
(12) Patent Application Publication
(10) Pub. No.: US 2021/0405054 A1
(43) Pub. Date: Dec. 30, 2021

(54) METHODS FOR DETECTING OVARIAN CANCER

(71) Applicant: Zora Biosciences Oy, Espoo (FI)

(72) Inventor: Mika HILVO, Helsinki (FI)

(21) Appl. No.: 16/625,142

(22) PCT Filed: Jul. 4, 2018
(86) PCT No.: PCT/EP2018/068043

§ 371 Date: Dec. 20, 2019

Related U.S. Application Data
(60) Provisional application No. 62/528,712, filed on Jul. 5, 2017.

(51) Int. Cl. G01N 33/574

(52) U.S. Cl. CPC G01N 33/57449; G01N 2800/50; G01N 2800/36; G01N 33/57484

(57) ABSTRACT

The present disclosure is related to the field of ovarian cancer diagnostics. It introduces novel biomarkers that can be used to detect presence of ovarian cancer and to provide a prognosis of the disease.

### FIELD OF THE INVENTION

The present description is related to the field of diagnostic and prognostic biomarkers for ovarian cancer. In particular, it provides a novel in vitro screening method for assessing whether a subject is at risk to develop or is suffering from ovarian cancer. In addition, the present biomarkers can be used in methods to evaluate survival prognosis, effectiveness of treatment and success of tumor removal of ovarian cancer.

### BACKGROUND

Ovarian cancer is a heterogeneous disease of low prevalence but poor survival. Early diagnosis is critical for the survival of the patient, as e.g. for the stage I patients the 5-year survival rate is around 90%, whereas for the stage IV patients it is only around 20%. However, the diagnosis of ovarian cancer is difficult, and the disease tends to cause symptoms for the patients only when advanced to later stages, and, in addition, the symptoms mimic often those of other diseases. Therefore new diagnostic tools that could detect ovarian cancer already in the early stages would be essential for successful treatment of ovarian cancer patients.

According to the World Health Organization (WHO) statistics in 2012, worldwide there were estimated to be 239 000 new ovarian cancer cases, representing 4% of all cancers in women. According to the statistics 152 000 deaths were caused by ovarian cancer in 2012. Ovarian cancer is the eighth most frequent cause of cancer death among women, and major proportion of new ovarian cancers occur in countries with high or very high levels of human development. (World Health Organization World Cancer Report 2014). The most common ovarian cancers are ovarian carcinomas, which include five main subtypes, and of those the high-grade serous carcinoma is the most common one (accounts approximately 70% of the cases).

Previous methods to detect ovarian cancer have relied on protein biomarker analyses and imaging methods. The main diagnostic methods for ovarian cancer at the moment include pelvic examination, CA-125 blood test and transvaginal ultrasound. CA-125 and HE4 are the only two biomarkers US Food and Drug Administration (FDA) approved for monitoring disease recurrence or progression, but not for screening. The multivariate index assay, OVA1, consisting of several protein biomarkers has been FDA approved for triage of pelvic masses since 2009. (Nguyen et al., Women's Health, 2013, 9(2), 171-187).

CA-125 has been reported to be a prognostic factor for overall and progression free survival in ovarian cancer, but studies showing contradictory results exist. CA-125 levels are raised in approximately 90% of patients with advanced epithelial ovarian cancer, but only in 50% of patients with stage I disease (Gupta & Lis, Journal of Ovarian Cancer, 2009, 2:13). Thus, the gold standard, CA-125 is relatively good in detecting patients with advanced disease, but it is lacking sensitivity in other patients. Consequently, its role in predicting survival is somewhat controversial.

Small molecules, including metabolites and lipids are tempting diagnostic tools in comparison to protein biomarkers, since they directly reflect changes in metabolism, which are known to occur early in tumor initiation and progression. Small changes in gene expression or protein levels of specific pathways may result in large changes in small molecule metabolite and lipid concentrations, as their levels can be considered to be an amplified output of the activity of the biological pathways.

Despite some attempts to find small molecule markers for ovarian cancer, however, most previous disclosures have failed to provide simple and reliable small molecule ovarian cancer biomarkers from blood serum that could be conveniently used in clinical practice. See e.g., WO2009151967, which describes a large panel of metabolic biomarker candidates and machine learning classification algorithms for ovarian cancer diagnostics. See also WO2012038525A1, which describes a large panel of phospholipids as biomarkers for various cancers and WO2013016700, which describes the use of classification algorithms to produce predictive models for epithelial ovarian cancer using data from mass spectrometry (MS) or nuclear magnetic resonance (NMR). Other potential biomarker candidates for ovarian cancer using the NMR technique is described e.g., in WO2011041892 and US2005170441.

Generally, the biomarker panels disclosed in the foregoing publications are too large for clinical use and there is no teaching regarding how the panels may be reduced in size. Further, it is unclear whether or not any of the disclosed metabolites or combinations thereof may be further combined with known protein markers to provide more accurate diagnostics and/or prognostics for ovarian cancer patients.

In contrast, WO/2016/051020, discloses small molecule biomarker panels having both diagnostic and prognostic value. Nevertheless, there is utility for additional, focused small molecule biomarker panels, which may be used to continue to improve the ability to assess, for example, a patient's risk of developing ovarian cancer, their prognosis and/or the expected effectiveness of a proposed treatment.

### SUMMARY OF THE INVENTION

The present invention identifies small molecule ovarian cancer biomarkers by quantifying defined molecular species and combinations thereof.

The present inventor has surprisingly found novel small molecule biomarker combinations for ovarian cancer. Specifically, it has been found that each marker displays a characteristic increase or decrease in concentration in samples derived from subjects having ovarian cancer, and they are useful for the methods and uses in accordance with the present disclosure. The present biomarkers are sensitive and specific and they can be used in diagnostic and prognostic assays. They are particularly useful for early stage ovarian cancer, such as ovarian cancer in stage I or II. Also, the marker combinations are especially useful in detecting ovarian cancer in premenopausal patients. In addition for ovarian cancer diagnosis most of the present biomarkers also associate with survival of the patients, and therefore they have a dual role in both predicting the patients with malignant ovarian tumors as well as predicting the prognosis for these patients. The present disclosure therefore represents a significant advantage over the prior methods currently used to diagnose and/or predict ovarian cancer.

Thus, the present small molecule biomarkers for ovarian cancer allow better diagnosis of or assessment of the risk to develop ovarian cancer. Further, the present markers find use in determining effectiveness of treatment and removal of tumors in patients having ovarian cancer. Further, the predictive or prognostic information from the small molecule biomarkers can be combined with protein biomarkers for ovarian cancer, such as CA-125, for early or late stage assessment.

For diagnostic use, a marker should have as high sensitivity and specificity as possible. Sensitivity measures the proportion of cases that are correctly classified as a case by the marker, and specificity measures the proportion of controls that are correctly classified as a control by the marker.

The two distinct classes of small molecules, Group A and Group B, that were found to provide improved diagnostic power when combined are shown in Table 1 below. According to all aspects of the present disclosure, the Group A and Group B small molecule biomarkers are selected from the small molecule biomarkers shown in Table 2. In some embodiments of all aspects of the present disclosure, the small molecule biomarkers are selected from the small molecule biomarkers shown in Table 3 or from the lipid biomarkers shown in Table 4.

TABLE 1
Small molecule biomarker groups A and B, having either increasing
or decreasing concentrations, respectively. Abbreviations are described
in the detailed description of the present disclosure.

| Group A | Group B |
|---|---|
| (Increasing | (Decreasing |
| concentration) | concentration) |
| Amino acids I | Amino acids II |
| Fatty acids | CE |
| Sugars | Ceramides with FA |
| Ceramides with FA | 14:0, 23:0, 24:0, 26:0 |
| 16:0, 18:0, 20:0, | DAG |
| 22:0, 24:1 | Gb3 |
| Butyric acids | Glc/GalCer |
| LPL I | LacCer |
| PE O I | LPL II |
| TAG I | PC |
| Other I | PC O/P |
| | PE |
| | PE O II |
| | PE P |
| | PG |
| | PI |
| | SM |
| | S1P/SA1P |
| | TAG II |
| | Other II |

According to a first aspect of the disclosure there is provided an in vitro screening method for assessing whether a subject is at risk to develop or is suffering from ovarian cancer comprising:
(a) assaying a sample from said subject to determine a concentration of at least one small molecule biomarker from Group A;
(b) assaying a sample from said subject to determine a concentration of at least one small molecule biomarker from Group B; and
(c) determining that the subject is suffering from or is at an increased risk of developing ovarian cancer, if said sample contains an increased concentration of the at least one small molecule biomarker from Group A and a decreased concentration of the at least one small molecule biomarker from Group B, when compared to a control.

In some embodiments, the method further comprises determining a level or concentration, of at least one protein biomarker for ovarian cancer in a sample, such as cancer antigen 125 (CA-125), human epididymal protein-4 (HE4), prealbumin, apolipoprotein A-1 (ApoA1), beta-2-microglobulin, transferrin (TRF), follicle-stimulating hormone (FSH) and CRP, and wherein an increase in CA-125, HE4, beta-2-microglobulin and/or CRP and/or a decrease in prealbumin, ApoA1, TRF and/or FSH level(s) or concentration(s) together with the presently identified small molecule biomarkers determination is indicative that the subject is at an increased risk of developing ovarian cancer or is suffering from ovarian cancer. Thus, the presently identified biomarkers can be used in combination with, for example, CA-125 and/or HE4 biomarker(s) to improve reliability of the determination by combining analysis of, for example, at least two small molecule biomarkers and, for example, CA-125 and/or HE4 biomarker(s). Furthermore, combined analysis enhances specificity and sensitivity of, for example, CA-125 and/or HE4 protein biomarker(s) for early and late stage ovarian cancer screening and prognosis. The protein biomarker(s) can be determined using any method known in the art. Typically, the determination of the protein biomarker(s) e.g., CA-125 and/or HE4 level(s) or concentration(s), is obtained from the same sample as used for determining the small molecule biomarker e.g., level(s) or concentration(s).

In some embodiments, the method for assessing whether a subject is at risk to develop or is suffering from ovarian cancer further comprises after the determining step (c), (d) diagnosing the subject as suffering from or having an increased risk of developing ovarian cancer from the results in step (c), and (e) administering a treatment to the subject diagnosed in step (d).

In one aspect, the present disclosure is directed to a method of treating or preventing ovarian cancer in a subject identified as being at risk to develop or suffering from ovarian cancer, the method comprising: administering to the subject a treatment as described herein, wherein prior to administering the treatment, the subject has been identified as being at risk to develop or suffering from ovarian cancer by the method described herein.

According to another aspect of the disclosure there is provided an in vitro screening method for assessing whether a premenopausal subject is at risk to develop or is suffering from ovarian cancer comprising:
(a) determining whether the subject is premenopausal;
(b) assaying a sample from said subject to determine a concentration of at least one small molecule biomarker from Group A;
(c) assaying a sample from said subject to determine a concentration of at least one small molecule biomarker from Group B; and
(d) determining that the subject is suffering from or is at an increased risk of developing ovarian cancer, if said sample contains an increased concentration of the at least one small molecule biomarker from Group A and a decreased concentration of the at least one small molecule biomarker from Group B, when compared to a control.

In some embodiments, the method further comprises determining a level or concentration of at least one protein biomarker for ovarian cancer in a sample, such as cancer antigen 125 (CA-125), human epididymal protein-4 (HE4), prealbumin, apolipoprotein A-1 (ApoA1), beta-2-microglobulin, transferrin (TRF), follicle-stimulating hormone (FSH) and CRP, wherein an increase in CA-125, HE4, beta-2-microglobulin and/or CRP and/or a decrease in prealbumin, ApoA1, TRF and/or FSH level(s) or concentration(s) together with the presently identified small molecule biomarkers determination is indicative that the premenopausal subject has an increased risk of developing or is suffering from ovarian cancer. Thus, the presently identified biomarkers can be used in combination with, for example, CA-125 and/or HE4 biomarker(s) to improve reliability of the determination by combining analysis of, for example, at least two small molecule biomarkers and, for example, CA-125 and/or HE4 biomarker(s). Furthermore, combined analysis enhances specificity and sensitivity of, for example, CA-125 and/or HE4 protein biomarker(s) for early and late stage ovarian cancer screening and prognosis. The protein biomarker(s) can be determined using any method known in the art. Typically, the determination of the protein biomarker(s) e.g., CA-125 and/or HE4 level(s) or concentration(s), is obtained from the same sample as used for determining the small molecule biomarker e.g., level(s) or concentration(s).

In some embodiments, the method for assessing whether a premenopausal subject is at risk to develop or is suffering from ovarian cancer further comprises after the determining step (d), (e) diagnosing the premenopausal subject as suffering from or having an increased risk of developing ovarian cancer from the results in step (d), and (f) administering a treatment to the subject diagnosed in step (e).

In one aspect, the present disclosure is directed to a method of treating or preventing ovarian cancer in a premenopausal subject identified as being at risk to develop or suffering from ovarian cancer, the method comprising: administering to the premenopausal subject a treatment as described herein, wherein prior to administering the treatment, the premenopausal subject has been identified as being at risk to develop or suffering from ovarian cancer by the method described herein.

According to another aspect of the disclosure there is provided a method of assessing whether a subject has a decreased or poor survival prognosis for ovarian cancer comprising:
(a) assaying a sample from said subject to determine a concentration of at least one small molecule biomarker from Group A;
(b) assaying a sample from said subject to determine a concentration of at least one small molecule biomarker from Group B; and
(c) determining that the subject has a decreased or poor survival prognosis, if said sample contains an increased concentration of the at least one small molecule biomarker from Group A and a decreased concentration of the at least one small molecule biomarker from Group B, when compared to a control.

Reliable prognosis may help an ovarian cancer patient to evaluate and select a particular treatment, duration and/or the intensity of any particular treatment including follow up treatments. Moreover, an accurate prognosis of ovarian cancer may be helpful to the wellbeing of a patient.

In some embodiments the method further comprises determining a level or concentration of at least one protein biomarker for ovarian cancer in a sample, such as cancer antigen 125 (CA-125), human epididymal protein-4 (HE4), prealbumin, apolipoprotein A-1 (ApoA1), beta-2-microglobulin, transferrin (TRF), follicle-stimulating hormone (FSH) and CRP, wherein an increase in CA-125, HE4, beta-2-microglobulin and/or CRP and/or a decrease in prealbumin, ApoA1, TRF and/or FSH level(s) or concentration(s) together with the presently identified small molecule biomarker(s) e.g., level(s) or concentration(s) may be used as an indicator of decreased or poor survival prognosis. Thus, the presently identified biomarkers can be used in combination with, for example, CA-125 and/or HE4 biomarker(s) to improve reliability of the determination by combining analysis of, for example, at least two small molecule biomarkers and, for example, CA-125 and/or HE4 biomarker(s). Furthermore, combined analysis enhances the prediction of the survival of ovarian cancer patients. The protein biomarker(s) can be determined using any method known in the art. Typically, the determination of the protein biomarker(s) e.g., CA-125 and/or HE4 level(s) or concentration(s) is obtained from the same sample as used for determining the small molecule biomarker e.g., level(s) or concentration(s).

In some embodiments, the method for assessing whether a subject has a decreased or poor survival prognosis for ovarian cancer further includes after the determining step (c), (d) administering a treatment to the subject.

In one aspect, the present disclosure is directed to a method of treating or preventing ovarian cancer in a subject identified as having a decreased or poor survival prognosis, the method comprising: administering to the subject a treatment as described herein, wherein prior to administering the treatment, the subject has been identified as having a decreased or poor survival prognosis for ovarian cancer by the method described herein.

According to yet another aspect of the disclosure there is provided an in vitro method for assessing the success rate of ovarian cancer tumor removal in a subject having received tumor therapy comprising:
(a) assaying a sample from said subject to determine a concentration of at least one small molecule biomarker from Group A;
(b) assaying a sample from said subject to determine a concentration of at least one small molecule biomarker from Group B; and
(c) determining that the tumor removal from the subject was successful, if said sample contains a decreased concentration of the at least one small molecule biomarker from Group A and an increased concentration of the at least one small molecule biomarker from Group B, when compared to a control.

In some embodiments, the method further comprises determining a level or concentration of at least one protein biomarker for ovarian cancer in a sample, such as cancer antigen 125 (CA-125), human epididymal protein-4 (HE4), prealbumin, apolipoprotein A-1 (ApoA1), beta-2-microglobulin, transferrin (TRF), follicle-stimulating hormone (FSH) and CRP, wherein a decrease in CA-125, HE4, beta-2-microglobulin and/or CRP and/or an increase in prealbumin, ApoA1, TRF and/or FSH level(s) or concentration(s) together with the presently identified small molecule biomarker(s) determination is indicative of successful tumor removal. Thus, the presently identified biomarkers can be used in combination with, for example, CA-125 and/or HE4 biomarker(s) to improve reliability of the determination of the tumor removal by combining analysis of, for example, at least two small molecule biomarkers and, for example, CA-125 and/or HE4 biomarker(s). The e.g., concentration(s) or level(s) of the protein biomarker(s) can be determined using any method known in the art. Typically, the determination of the protein biomarker(s) e.g., CA-125 and/or HE4 level(s) or concentration(s), is obtained from the same sample as used for determining the small molecule biomarker e.g., level(s) or concentration(s).

Monitoring the tumor removal success is an important aspect of a treatment and it provides valuable information about the need for further operations or treatments. Accordingly, a treatment may be followed by further operations or drug therapy based on the result from the above assessment.

In some embodiments, the method for assessing the success rate of ovarian cancer tumor removal in a subject having received tumor therapy further comprises, (d) determining that the tumor removal was not successful from the results in step (c), and (e) administering a further treatment to the subject.

In one aspect, the present disclosure is directed to a method of treating ovarian cancer in a subject identified as having received an unsuccessful tumor removal, the method comprising: administering to the subject a further treatment as described herein, wherein prior to administering the treatment, the subject has been identified as having received an unsuccessful tumor removal by the method described herein.

According to yet another aspect of the invention there is provided an in vitro method of evaluating the effectiveness of an ovarian cancer therapy in a subject comprising:
(a) assaying a sample from said subject to determine a concentration of at least one small molecule biomarker from Group A;
(b) assaying a sample from said subject to determine a concentration of at least one small molecule biomarker from Group B; and
(c) determining that the therapy is effective, if said sample contains a decreased concentration of the at least one small molecule biomarker from Group A and an increased concentration of the at least one small molecule biomarker from Group B, when compared to a control.

In the method, the effectiveness of the therapy is monitored by analysing the concentrations of the presently identified small molecule biomarkers. The concentrations of the selected biomarkers reflect the progress of the ovarian cancer and their concentrations approach the concentrations determined for the control when the cancer responds to said therapy. Accordingly, the therapy may be tailored based on the subject such that only therapy which shows a positive response, and is thus found effective, is continued, and therapy which shows no response, and is thus found ineffective, is discontinued.

In some embodiments, the method further comprises determining a level or concentration of at least one protein biomarker for ovarian cancer in a sample, such as cancer antigen 125 (CA-125), human epididymal protein-4 (HE4), prealbumin, apolipoprotein A-1 (ApoA1), beta-2-microglobulin, transferrin (TRF), follicle-stimulating hormone (FSH) and CRP, wherein a decrease in CA-125, HE4, beta-2-microglobulin and/or CRP and/or an increase in prealbumin, ApoA1, TRF and/or FSH level(s) or concentration(s) together with the presently identified small molecule biomarkers determination is indicative for an effective therapy. Thus, the presently identified biomarkers can be used in combination with, for example, CA125 and/or HE4 biomarker(s) to improve reliability of the determination of the therapy effectiveness by combining analysis, for example, at least two small molecule biomarkers and, for example, CA-125 and/or HE4 biomarker(s). The protein biomarker(s) can be determined using any method known in the art. Typically, the determination of the protein biomarker(s) e.g., CA-125 and/or HE4 level(s) or concentration(s), is obtained from the same sample as used for determining the small molecule biomarker e.g., level(s) or concentration(s).

In some embodiments, the method for evaluating the effectiveness of an ovarian cancer therapy in a subject further comprises, (d) determining that the therapy is not effective in the subject from the results in step (c), and (e) escalating the therapy of the subject.

In one aspect, the present disclosure is directed to a method of treating ovarian cancer in a subject identified as being ineffectively treated, the method comprising: administering to the subject a further treatment as described herein, wherein prior to administering the treatment, the subject has been identified as being ineffectively treated for ovarian cancer by the methods described herein.

According to yet another aspect of the disclosure there is provided an in vitro screening method for assessing whether a subject is at risk to develop or is suffering from ovarian cancer comprising:
(a) assaying a sample from said subject to determine a concentration of cancer antigen 125 (CA-125);
(b) assaying a sample from said subject to determine a concentration of at least one small molecule biomarker from Group B; and
(c) determining that the subject is suffering from or is at an increased risk of developing ovarian cancer, if said sample contains an increased concentration of CA-125 and a decreased concentration of the at least one small molecule biomarker from Group B, when compared to a control.

In some embodiments, the method further comprises determining a level or concentration, of at least one additional protein biomarker for ovarian cancer in a sample, such as human epididymal protein-4 (HE4), prealbumin, apolipoprotein A-1 (ApoA1), beta-2-microglobulin, transferrin (TRF), follicle-stimulating hormone (FSH) and CRP, and wherein an increase in HE4, beta-2-microglobulin and/or CRP and/or a decrease in prealbumin, ApoA1, TRF and/or FSH level(s) or concentration(s) together with the presently identified CA-125 and small molecule biomarkers determination is indicative that the subject is at an increased risk of developing ovarian cancer or is suffering from ovarian cancer. Thus, the presently identified biomarkers can be used in combination with, for example, CA-125 and/or HE4 biomarker(s) to improve reliability of the determination by combining analysis of, for example, at least two small molecule biomarkers and, for example, CA-125 and/or HE4 biomarker(s). Furthermore, combined analysis enhances specificity and sensitivity of, for example, CA-125 and/or HE4 protein biomarker(s) for early and late stage ovarian cancer screening and prognosis. The protein biomarker(s) can be determined using any method known in the art. Typically, the determination of the protein biomarker(s) e.g., CA-125 and/or HE4 level(s) or concentration(s), is obtained from the same sample as used for determining the small molecule biomarker e.g., level(s) or concentration(s).

In some embodiments, the method for assessing whether a subject is at risk to develop or is suffering from ovarian cancer further comprises after the determining step (c), (d) diagnosing the subject as suffering from or having an increased risk of developing ovarian cancer from the results in step (c), and (e) administering a treatment to the subject diagnosed in step (d).

In one aspect, the present disclosure is directed to a method of treating or preventing ovarian cancer in a subject identified as being at risk to develop or suffering from ovarian cancer, the method comprising: administering to the subject a treatment as described herein, wherein prior to administering the treatment, the subject has been identified as being at risk to develop or suffering from ovarian cancer by the method described herein.

According to yet another aspect of the disclosure there is provided an in vitro screening method for assessing whether a premenopausal subject is at risk to develop or is suffering from ovarian cancer comprising:
(a) determining whether the subject is premenopausal;
(b) assaying a sample from said subject to determine a concentration of cancer antigen 125 (CA-125);
(c) assaying a sample from said subject to determine a concentration of at least one small molecule biomarker from Group B; and
(d) determining that the subject is suffering from or is at an increased risk of developing ovarian cancer, if said sample contains an increased concentration of CA-125 and a decreased concentration of the at least one small molecule biomarker from Group B, when compared to a control.

In some embodiments, the method further comprises determining a level or concentration of at least one additional protein biomarker for ovarian cancer in a sample, such as human epididymal protein-4 (HE4), prealbumin, apolipoprotein A-1 (ApoA1), beta-2-microglobulin, transferrin (TRF), follicle-stimulating hormone (FSH) and CRP, wherein an increase in HE4, beta-2-microglobulin and/or CRP and/or a decrease in prealbumin, ApoA1, TRF and/or FSH level(s) or concentration(s) together with the presently identified CA-125 and small molecule biomarkers determination is indicative that the premenopausal subject has an increased risk of developing or is suffering from ovarian cancer. Thus, the presently identified biomarkers can be used in combination with, for example, CA-125 and/or HE4 biomarker(s) to improve reliability of the determination by combining analysis of, for example, at least two small molecule biomarkers and, for example, CA-125 and/or HE4 biomarker(s). Furthermore, combined analysis enhances specificity and sensitivity of, for example, CA-125 and/or HE4 protein biomarker(s) for early and late stage ovarian cancer screening and prognosis. The protein biomarker(s) can be determined using any method known in the art. Typically, the determination of the protein biomarker(s) e.g., CA-125 and/or HE4 level(s) or concentration(s), is obtained from the same sample as used for determining the small molecule biomarker e.g., level(s) or concentration(s).

In some embodiments, the method for assessing whether a premenopausal subject is at risk to develop or is suffering from ovarian cancer further comprises after the determining step (d), (e) diagnosing the premenopausal subject as suffering from or having an increased risk of developing ovarian cancer from the results in step (d), and (f) administering a treatment to the subject diagnosed in step (e).

In one aspect, the present disclosure is directed to a method of treating or preventing ovarian cancer in a premenopausal subject identified as being at risk to develop or suffering from ovarian cancer, the method comprising: administering to the premenopausal subject a treatment as described herein, wherein prior to administering the treatment, the premenopausal subject has been identified as being at risk to develop or suffering from ovarian cancer by the method described herein.

In some embodiments, the therapy may comprise any therapeutic treatment or operations typically given to a subject having ovarian cancer, such as, but not limited to, surgery, chemotherapy, radiation therapy, hormonal therapy, anti-angiogenic therapy, therapies targeting homologous recombination deficiency, antibody therapy or other targeted therapy utilizing ovarian cancer specific signalling pathways. The treatment may comprise treatment having a direct effect on the metabolism of the malignant tissue. Ovarian cancer therapy may comprise administering a pharmaceutical agent affecting lipid metabolism.

In some embodiments, the ovarian cancer patient is capable of being treated with at least one of surgery, chemotherapy, radiation therapy, hormonal therapy, anti-angiogenic therapy, therapies targeting homologous recombination deficiency, antibody therapy or other targeted therapy utilizing ovarian cancer specific signalling pathways. The treatment may include a treatment having a direct effect on the metabolism of the malignant tissue. As used herein “capable of being treated” means that the treatment and/or therapy is not contraindicated in the patient because of e.g., age of the patient, stage of the ovarian cancer and/or other diseases or conditions.

In some embodiments of the aforementioned methods, the method of treating ovarian cancer further comprises identifying the subject as in need of the treatment or prevention, for example, requesting a test or receiving the test results, for example, from a commercial laboratory, which provides the results of an assay useful for determining the concentration of the at least one small molecule biomarker from Group A and the at least one small molecule biomarker from Group B and administering to the subject a treatment, for example, a therapeutically effective dose of a drug, if the subject has an increased concentration of the at least one small molecule biomarker from Group A and a decreased concentration of the at least one small molecule biomarker from Group B, as compared to the control.

According to another aspect of the disclosure there is provided a method of detecting in a sample obtained from a subject the concentration of at least one small molecule biomarker from Group A and at least one small molecule biomarker from Group B comprising:
(a) assaying the sample from said subject to determine the concentration of the at least one small molecule biomarker from Group A;
(b) assaying the sample from said subject to determine the concentration of the at least one small molecule biomarker from Group B; and optionally
(c) comparing the concentration of the at least one small molecule biomarker from Group A and the concentration of the at least one small molecule biomarker from Group B to a control.

In some embodiments of the aforementioned method of detecting, the method further comprises determining a level or concentration of at least one protein biomarker for ovarian cancer in a sample, such as cancer antigen 125 (CA-125), human epididymal protein-4 (HE4), prealbumin, apolipoprotein A-1 (ApoA1), beta-2-microglobulin, transferrin (TRF), follicle-stimulating hormone (FSH) and CRP. The protein biomarker(s) e.g., level(s) or concentration(s) can be determined using any method known in the art. Typically, the determination of the protein biomarker(s), for example, CA-125 and/or HE4 level(s) or concentration(s) is obtained from the same sample as used for determining the small molecule biomarker e.g., level(s) or concentration(s).

According to yet another aspect of the disclosure there is provided a method of detecting in a sample obtained from a subject the concentration of cancer antigen 125 (CA-125) and at least one small molecule biomarker from Group B comprising:
(a) assaying the sample from said subject to determine the concentration of CA-125;
(b) assaying the sample from said subject to determine the concentration of the at least one small molecule biomarker from Group B; and optionally
(c) comparing the concentration of CA-125 and the concentration of the at least one small molecule biomarker from Group B to a control.

In some embodiments of the aforementioned method of detecting, the method further comprises determining a level or concentration of at least one additional protein biomarker for ovarian cancer in a sample, such as human epididymal protein-4 (HE4), prealbumin, apolipoprotein A-1 (ApoA1), beta-2-microglobulin, transferrin (TRF), follicle-stimulating hormone (FSH) and CRP. The protein biomarker(s) e.g., level(s) or concentration(s) can be determined using any method known in the art. Typically, the determination of the protein biomarker(s), for example, CA-125 and/or HE4 level(s) or concentration(s) is obtained from the same sample as used for determining the small molecule biomarker e.g., level(s) or concentration(s).

In some embodiments, the subject used in the foregoing detecting methods is a female subject, typically a postmenopausal female subject, more typically a premenopausal female subject.

According to another aspect of the disclosure, there is provided a method of collecting data for assessing whether a subject is at risk to develop or is suffering from ovarian cancer comprising:
(a) assaying a sample from said subject to determine a concentration of at least one small molecule biomarker from Group A;
(b) assaying a sample from said subject to determine a concentration of at least one small molecule biomarker from Group B; and
(c) determining that the subject is suffering from or is at an increased risk of developing ovarian cancer, if said sample contains an increased concentration of the at least one small molecule biomarker from Group A and a decreased concentration of the at least one small molecule biomarker from Group B, when compared to a control.

According to another aspect of the disclosure there is provided a method of collecting data for assessing whether a premenopausal subject is at risk to develop or is suffering from ovarian cancer comprising:
(a) determining whether the subject is premenopausal;
(b) assaying a sample from said subject to determine a concentration of at least one small molecule biomarker from Group A;
(c) assaying a sample from said subject to determine a concentration of at least one small molecule biomarker from Group B; and
(d) determining that the subject is suffering from or is at an increased risk of developing ovarian cancer, if said sample contains an increased concentration of the at least one small molecule biomarker from Group A and a decreased concentration of the at least one small molecule biomarker from Group B, when compared to a control.

According to yet another aspect of the disclosure, there is provided a method of collecting data for assessing whether a subject has a decreased or poor survival prognosis for ovarian cancer comprising:
(a) assaying a sample from said subject to determine a concentration of at least one small molecule biomarker from Group A;
(b) assaying a sample from said subject to determine a concentration of at least one small molecule biomarker from Group B; and
(c) determining that the subject has a decreased or poor survival prognosis, if said sample contains an increased concentration of the at least one small molecule biomarker from Group A and a decreased concentration of the at least one small molecule biomarker from Group B, when compared to a control.

According to yet another aspect of the disclosure, there is provided a method of collecting data for assessing the success rate of ovarian cancer tumor removal in a subject having received tumor therapy comprising:
(a) assaying a sample from said subject to determine a concentration of at least one small molecule biomarker from Group A;
(b) assaying a sample from said subject to determine a concentration of at least one small molecule biomarker from Group B; and
(c) determining that the tumor removal from the subject was successful, if said sample contains a decreased concentration of the at least one small molecule biomarker from Group A and an increased concentration of the at least one small molecule biomarker from Group B, when compared to a control.

According to yet another aspect of the invention there is provided a method of collecting data for evaluating the effectiveness of an ovarian cancer therapy in a subject comprising:
(a) assaying a sample from said subject to determine a concentration of at least one small molecule biomarker from Group A;
(b) assaying a sample from said subject to determine a concentration of at least one small molecule biomarker from Group B; and
(c) determining that the therapy is effective, if said sample contains a decreased concentration of the at least one small molecule biomarker from Group A and an increased concentration of the at least one small molecule biomarker from Group B, when compared to a control.

In some embodiments of the aforementioned methods of collecting data, the methods further comprise determining a level or concentration of at least one protein biomarker for ovarian cancer in a sample, such as cancer antigen 125 (CA-125), human epididymal protein-4 (HE4), prealbumin, apolipoprotein A-1 (ApoA1), beta-2-microglobulin, transferrin (TRF), follicle-stimulating hormone (FSH) and CRP. Thus, the presently identified biomarkers can be used in combination with, for example, CA-125 and/or HE4 biomarker(s) to improve the performance of the methods by combining analysis of, for example, at least two small molecule biomarkers and, for example, CA-125 and/or HE4 biomarker(s). The protein biomarker(s) can be determined using any method known in the art. Typically, the determination of the protein biomarker(s) e.g., CA-125 and/or HE4 level(s) or concentration(s), is obtained from the same sample as used for determining the small molecule biomarker e.g., level(s) or concentration(s).

According to yet another aspect of the disclosure, there is provided a method of collecting data for assessing whether a subject is at risk to develop or is suffering from ovarian cancer comprising:
(a) assaying a sample from said subject to determine a concentration of cancer antigen 125 (CA-125);
(b) assaying a sample from said subject to determine a concentration of at least one small molecule biomarker from Group B; and
(c) determining that the subject is suffering from or is at an increased risk of developing ovarian cancer, if said sample contains an increased concentration of CA-125 and a decreased concentration of the at least one small molecule biomarker from Group B, when compared to a control.

According to yet another aspect of the disclosure there is provided a method of collecting data for assessing whether a premenopausal subject is at risk to develop or is suffering from ovarian cancer comprising:
(a) determining whether the subject is premenopausal;
(b) assaying a sample from said subject to determine a concentration of cancer antigen 125 (CA-125);
(c) assaying a sample from said subject to determine a concentration of at least one small molecule biomarker from Group B; and
(d) determining that the subject is suffering from or is at an increased risk of developing ovarian cancer, if said sample contains an increased concentration of CA-125 and a decreased concentration of the at least one small molecule biomarker from Group B, when compared to a control.

In some embodiments of the aforementioned methods of collecting data, the methods further comprise determining a level or concentration of at least one additional protein biomarker for ovarian cancer in a sample, such as human epididymal protein-4 (HE4), prealbumin, apolipoprotein A-1 (ApoA1), beta-2-microglobulin, transferrin (TRF), follicle-stimulating hormone (FSH) and CRP. Thus, the presently identified biomarkers can be used in combination with, for example, CA-125 and/or HE4 biomarker(s) to improve the performance of the methods by combining analysis of, for example, at least two small molecule biomarkers and, for example, CA-125 and/or HE4 biomarker(s). The protein biomarker(s) can be determined using any method known in the art. Typically, the determination of the protein biomarker(s) e.g., CA-125 and/or HE4 level(s) or concentration(s), is obtained from the same sample as used for determining the small molecule biomarker e.g., level(s) or concentration(s).

Yet another aspect of the present disclosure is a composition or kit for diagnosing, predicting or detecting ovarian cancer or for performing any of the methods or uses disclosed herein. In some embodiments, the composition or kit comprises at least one small molecule biomarker from Group A and at least one small molecule biomarker from Group B. In other embodiments, the composition or kit comprises at least one isotope (e.g. deuterium)-labelled small molecule biomarker from Group A and at least one isotope (e.g. deuterium)-labelled small molecule biomarker from Group B.

In other embodiments, the composition or kit comprises at least 1, at least 2, at least 3, at least 4, at least 5, at least 6, at least 7, at least 8, at least 9 or at least 10 small molecule biomarkers from Group A and at least 1, at least 2, at least 3, at least 4, at least 5, at least 6, at least 7, at least 8, at least 9 or at least 10 small molecule biomarkers from Group B.

In yet other embodiments, the composition or kit comprises at least 1, at least 2, at least 3, at least 4, at least 5, at least 6, at least 7, at least 8, at least 9 or at least 10 isotope (e.g. deuterium)-labelled small molecule biomarkers from Group A and at least 1, at least 2, at least 3, at least 4, at least 5, at least 6, at least 7, at least 8, at least 9 or at least 10 isotope (e.g. deuterium)-labelled small molecule biomarkers from Group B.

In other embodiments, the composition or kit includes at least one protein biomarker for ovarian cancer, such as cancer antigen 125 (CA-125), human epididymal protein-4 (HE4), prealbumin, apolipoprotein A-1 (ApoA1), beta-2-microglobulin, transferrin (TRF), follicle-stimulating hormone (FSH) and CRP.

Yet another aspect of the present disclosure is a composition or kit for diagnosing, predicting or detecting ovarian cancer or for performing any of the methods or uses disclosed herein. In some embodiments, the composition or kit comprises cancer antigen 125 (CA-125) and at least one small molecule biomarker from Group B. In other embodiments, the composition or kit comprises at least one isotope (e.g. deuterium)-labelled CA-125 and at least one isotope (e.g. deuterium)-labelled small molecule biomarker from Group B.

In some embodiments, the composition or kit comprises at least 1, at least 2, at least 3, at least 4, at least 5, at least 6, at least 7, at least 8, at least 9 or at least 10 small molecule biomarkers from Group B.

In other embodiments, the composition or kit comprises at least 1, at least 2, at least 3, at least 4, at least 5, at least 6, at least 7, at least 8, at least 9 or at least 10 isotope (e.g. deuterium)-labelled small molecule biomarkers from Group B.

In other embodiments, the composition or kit includes at least one additional protein biomarker for ovarian cancer, such as human epididymal protein-4 (HE4), prealbumin, apolipoprotein A-1 (ApoA1), beta-2-microglobulin, transferrin (TRF), follicle-stimulating hormone (FSH) and CRP.

The composition or kit may further include standards, controls, reagents, solutions, solvents, containers, instructions to use it for the methods or uses disclosed herein or other elements for performing the methods or uses disclosed herein.

In some embodiments, the composition or kit includes elements for collecting a blood sample, for example, a dried blood spot on a filter.

The composition or kit may be a test kit for used in a laboratory or a home use test kit (overthe-counter test). The composition or kit may be used in assays performed with various chemical and high-resolution analytical techniques, as appropriate. Suitable analytical techniques according to the present methods and uses include, but are not limited to, mass spectrometry (MS) and nuclear magnetic resonance (NMR). Any high-resolution technique capable of resolving individual small molecule biomarkers can be used to collect the information on the biomarker in question, such as the concentration of biomarker profile from the biological sample. Typically, the information is collected using mass spectrometry. The MS analysis can be coupled to another high performance separation method, such as gas chromatography (GC), two-dimensional gas chromatography (GC×GC), liquid chromatography (LC), high performance liquid chromatography (HPLC) or ultra performance liquid chromatography (U PLC).

According to another aspect of the disclosure there is provided a use of one or more reagent(s) in the manufacture of a test, kit, composition, preparation or medicament for assessing whether a subject is at risk to develop or is suffering from ovarian cancer comprising:
(a) a reagent for assaying a sample from said subject to determine a concentration of at least one small molecule biomarker from Group A;
(b) a reagent for assaying a sample from said subject to determine a concentration of at least one small molecule biomarker from Group B; and
(c) determining that the subject is suffering from or is at an increased risk of developing ovarian cancer, if said sample contains an increased concentration of the at least one small molecule biomarker from Group A and a decreased concentration of the at least one small molecule biomarker from Group B, when compared to a control.

According to another aspect of the disclosure there is provided a use of one or more reagent(s) in the manufacture of a test, kit, composition, preparation or medicament for assessing whether a premenopausal subject is at risk to develop or is suffering from ovarian cancer comprising:
(a) determining whether the subject is premenopausal;
(b) a reagent for assaying a sample from said subject to determine a concentration of at least one small molecule biomarker from Group A;
(c) a reagent for assaying a sample from said subject to determine a concentration of at least one small molecule biomarker from Group B; and
(d) determining that the subject is suffering from or is at an increased risk of developing ovarian cancer, if said sample contains an increased concentration of the at least one small molecule biomarker from Group A and a decreased concentration of the at least one small molecule biomarker from Group B, when compared to a control.

According to yet another aspect of the disclosure there is provided a use of one or more reagent(s) in the manufacture of a test, kit, composition, preparation or medicament for assessing whether a subject has a decreased or poor survival prognosis for ovarian cancer comprising:
(a) a reagent for assaying a sample from said subject to determine a concentration of at least one small molecule biomarker from Group A;
(b) a reagent for assaying a sample from said subject to determine a concentration of at least one small molecule biomarker from Group B; and
(c) determining that the subject is having a decreased or poor survival prognosis, if said sample contains an increased concentration of the at least one small molecule biomarker from Group A and a decreased concentration of the at least one small molecule biomarker from Group B, when compared to a control.

According to yet another aspect of the disclosure there is provided a use of one or more reagent(s) in the manufacture of a test, kit, composition, preparation or medicament for assessing the success rate of ovarian cancer tumor removal in a subject having received tumor therapy comprising:
(a) a reagent for assaying a sample from said subject to determine a concentration of at least one small molecule biomarker from Group A;
(b) a reagent for assaying a sample from said subject to determine a concentration of at least one small molecule biomarker from Group B; and
(c) determining that the tumor removal from the subject was successful, if said sample contains a decreased concentration of the at least one small molecule biomarker from Group A and an increased concentration of the at least one small molecule biomarker from Group B, when compared to a control.

According to yet another aspect of the disclosure there is provided a use of one or more reagent(s) in the manufacture of a test, kit, composition, preparation or medicament for evaluating the effectiveness of an ovarian cancer therapy in a subject comprising:
(a) a reagent for assaying a sample from said subject to determine a concentration of at least one small molecule biomarker from Group A;
(b) a reagent for assaying a sample from said subject to determine a concentration of at least one small molecule biomarker from Group B; and
(c) determining that the therapy is effective, if said sample contains a decreased concentration of the at least one small molecule biomarker from Group A and an increased concentration of the at least one small molecule biomarker from Group B, when compared to a control.

In some embodiments of the aforementioned uses of one or more reagent(s) in the manufacture of a test, kit, composition, preparation or medicament, the uses further comprise one or more reagent(s) for determining a level or concentration of at least one protein biomarker for ovarian cancer in a sample, such as cancer antigen 125 (CA-125), human epididymal protein-4 (HE4), prealbumin, apolipoprotein A-1 (ApoA1), beta-2-microglobulin, transferrin (TRF), follicle-stimulating hormone (FSH) and CRP. Thus, the presently identified biomarkers can be used in combination with, for example, CA-125 and/or HE4 biomarker(s) to improve the performance of the uses by combining analysis of, for example, at least two small molecule biomarkers and, for example, CA-125 and/or HE4 biomarker(s). The protein biomarker(s) can be determined using any method known in the art. Typically, the determination of the protein biomarker(s) e.g., CA-125 and/or HE4 level(s) or concentration(s), is obtained from the same sample as used for determining the small molecule biomarker e.g., level(s) or concentration(s).

According to yet another aspect of the disclosure there is provided a use of one or more reagent(s) in the manufacture of a test, kit, composition, preparation or medicament for assessing whether a subject is at risk to develop or is suffering from ovarian cancer comprising:
(a) a reagent for assaying a sample from said subject to determine a concentration of cancer antigen 125 (CA-125);
(b) a reagent for assaying a sample from said subject to determine a concentration of at least one small molecule biomarker from Group B; and
(c) determining that the subject is suffering from or is at an increased risk of developing ovarian cancer, if said sample contains an increased concentration of CA-125 and a decreased concentration of the at least one small molecule biomarker from Group B, when compared to a control.

According to yet another aspect of the disclosure there is provided a use of one or more reagent(s) in the manufacture of a test, kit, composition, preparation or medicament for assessing whether a premenopausal subject is at risk to develop or is suffering from ovarian cancer comprising:
(a) determining whether the subject is premenopausal;
(b) a reagent for assaying a sample from said subject to determine a concentration of cancer antigen 125 (CA-125);
(c) a reagent for assaying a sample from said subject to determine a concentration of at least one small molecule biomarker from Group B; and
(d) determining that the subject is suffering from or is at an increased risk of developing ovarian cancer, if said sample contains an increased concentration of CA-125 and a decreased concentration of the at least one small molecule biomarker from Group B, when compared to a control.

In some embodiments of the aforementioned uses of one or more reagent(s) in the manufacture of a test, kit, composition, preparation or medicament, the uses further comprise one or more reagent(s) for determining a level or concentration of at least one additional protein biomarker for ovarian cancer in a sample, such as human epididymal protein-4 (HE4), prealbumin, apolipoprotein A-1 (ApoA1), beta-2-microglobulin, transferrin (TRF), follicle-stimulating hormone (FSH) and CRP. Thus, the presently identified biomarkers can be used in combination with, for example, CA-125 and/or HE4 biomarker(s) to improve the performance of the uses by combining analysis of, for example, at least two small molecule biomarkers and, for example, CA-125 and/or HE4 biomarker(s). The protein biomarker(s) can be determined using any method known in the art. Typically, the determination of the protein biomarker(s) e.g., CA-125 and/or HE4 level(s) or concentration(s), is obtained from the same sample as used for determining the small molecule biomarker e.g., level(s) or concentration(s).

The determination of the biomarkers is typically performed using an assay. The assay can be performed with various chemical and high-resolution analytical techniques, as appropriate. Suitable analytical techniques according to the present methods and uses include, but are not limited to, mass spectrometry (MS) and nuclear magnetic resonance (NMR). Any high-resolution technique capable of resolving individual small molecule biomarkers can be used to collect the information on the biomarker in question, such as the concentration of biomarker profile from the biological sample, such as blood, blood serum, blood plasma, tissue, urine or saliva. Typically, the information is collected using mass spectrometry. The MS analysis can be coupled to another high performance separation method, such as gas chromatography (GC), two-dimensional gas chromatography (GC×GC), liquid chromatography (LC), high performance liquid chromatography (HPLC) or ultra performance liquid chromatography (U PLC).

The sample from the subject and the control sample may be a blood sample, a blood serum sample, a blood plasma sample, a saliva sample or an urine sample. Blood serum and plasma samples are typically used. The sample can be prepared with techniques well known in the art. Alternatively, both the sample from the subject and the control sample may also be tissue samples, e.g., ovarian tissue sample.

According to certain embodiments, the methods and uses of the disclosure provide for measuring the levels of small molecule biomarkers in a plasma sample or a serum sample without the need to isolate or enrich exfoliated tumor cells in said sample prior to detection. In some embodiments, the sample is a non-sedimented sample. In other embodiments, the plasma sample is substantially free of residual cells. In yet other embodiments, the blood sample is treated with clot activators and serum is separated by centrifugation, optionally followed by freezing and thawing, prior to analysis.

In some embodiments, before the mass spectrometric analysis, the small molecule biomarkers of the sample are extracted with a solvent or a solvent mixture from the sample. Suitable solvents include organic solvents such as methanol, chloroform/methanol or other similar solvents.

In other embodiments, the small molecule biomarkers are derivatized before the mass spectrometric analysis. In some embodiments, the derivatization comprises extraction with an organic solvent, evaporation of the solvent under reduced pressure, and derivatization.

In other embodiments, the sample is filtered before determining the small molecule biomarkers by using a filter which removes cells. In other embodiments, a filter having a cut-off value of 30 kDa is used to remove cells. In yet other embodiments, a filter is used which removes proteins. In other embodiments, the sample is reconstituted after the filtering.

In other embodiments, the sample or the reconstituted sample is diluted prior to determining the small molecule biomarkers. In other embodiments, the sample is diluted at least 1:2 before determining the small molecule biomarkers.

In other embodiments, a preservative or an internal mass standard is added to the sample.

In yet other embodiments of the methods and uses of the present disclosure, the methods and uses further comprise a step of spiking the sample with at least one isotope-labelled small molecule biomarker from Group A and/or at least one isotope-labelled small molecule biomarker from Group B prior to determining the concentration of the at least one small molecule biomarker from Group A and the concentration of the at least one small molecule biomarker from Group B. The at least one isotope-labelled small molecule biomarker from Group A and/or the at least one isotope-labelled small molecule biomarker from Group B may be, but is not limited to, deuterium-labelled small molecule biomarker from Group A and/or Group B.

The small molecule ovarian cancer biomarkers of the present disclosure allow for easy, reliable and early prediction of ovarian cancer. This will facilitate e.g. earlier intervention, less symptom development and suffering and decreased morbidity. The present biomarkers also allow easy monitoring of the progress of the ovarian cancer as the analysis can be performed on, for example, serum or plasma samples without the need of collecting a tissue sample.

In certain embodiments, the ovarian cancer is an early stage ovarian cancer.

As described elsewhere in the present disclosure, the control may be a concentration determined from a single healthy individual or a subject with benign tumor or other medical condition causing similar symptoms to ovarian cancer, or the same subject before developing malignant tissue. The control may also be a sample that represents a combination of samples from a generalized population of healthy individuals. Alternatively, the control may be a control value or a set of data concerning the biomarker in a sample previously determined, calculated or extrapolated, or may have yet to be determined, calculated or extrapolated, or may also be taken from the literature.

In typical embodiments, the above methods and uses comprise determining the concentration of at least 1, at least 2, at least 3, at least 4, at least 5, at least 6, at least 7, at least 8, at least 9, at least 10 or more small molecule biomarkers from Group A and at least 1, at least 2, at least 3, at least 4, at least 5, at least 6, at least 7, at least 8, at least 9, at least 10 or more small molecule biomarkers from Group B.

In typical embodiments, the biomarker concentration is determined by using mass spectrometry, nuclear magnetic resonance spectroscopy, fluorescence spectroscopy or dual polarization interferometry, an immunoassay, enzymatic assay, colorimetric assay, fluorometric assay, a rapid test, a breath test and/or with a binding moiety capable of specifically binding the biomarker.

In another aspect, there is provided use of a pharmaceutical for treating ovarian cancer or one or more of its complications in a subject in need thereof, the use comprising using an effective pharmaceutical for ovarian cancer treatment, wherein the effectiveness of treatment with the pharmaceutical is evaluated using the aforementioned method for evaluating the effectiveness of an ovarian cancer therapy.

Typically, the pharmaceutical is administered at a dose which causes the concentration of the at least one small molecule ovarian cancer biomarker in the sample to change from the initial level towards the concentration in the control, and wherein the concentration of the at least one small molecule ovarian cancer biomarker is determined according to the aforementioned methods or uses.

### DETAILED DESCRIPTION OF THE INVENTION

Reference will now be made in detail to various exemplary embodiments, examples of which are discussed in the detailed description that follows. It is to be understood that the following detailed description is provided to give the reader a fuller understanding of certain embodiments, features, and details of aspects of the invention, and should not be interpreted as limiting the scope of the invention.

### Abbreviations

Unless indicated otherwise, the abbreviations used in this description have the following meanings: ApoA1—apolipoprotein A1, AUC—area under the curve, BSTFA—N,O-bis(trimethylsilyl)trifluoroacetamide, CA-125—cancer antigen 125, carcinoma antigen 125, carbohydrate antigen 125, CE—cholesterylester, Cer—ceramide, CI—confidence interval, CRP—C-reactive protein, DAG—diacylglycerol, EI—electron ionization, FA—fatty acid, FDA—US Food and Drug Administration, FSH—follicle-stimulating hormone, Gb3 globotriasoylceramide, GC—gas chromatography, GC×GC—two dimensional gas chromatography, GIc/GalCer—glucosyl/galactosylceramide, HE4—human epididymic protein 4, HPLC—high performance liquid chromatography, HR—hazard ratio, IS—internal standard, LacCer—lactosylceramide, LC—liquid chromatography, LPC—lyosphosphatidylcholine, LPC-O—ether-linked (alkyl) lysophosphatidylcholine, LPE—lysophosphatidylethanolamine, LPL—lysophospholipid, MRM—multiple reaction monitoring, MS—mass spectrometry, NMR—nuclear magnetic resonance, PC—phosphatidylcholine, PC O—ether-linked (alkyl) phosphatidylcholine, PC P—ether-linked (alkenyl) phosphatidylcholine, PE—phosphatidylethanolamine, PE O—ether-linked (alkyl) phosphatidylethanolamine, PE P—ether-linked (alkenyl) phosphatidylethanolamine, PG—phosphatidylglycerol, PI—phosphatidylinositol, ROC—receiver operating characteristic, S1P—sphingosine-1-phosphate, SA1P—sphinganine-1-phosphate, SE—sensitivity, SIM—selected ion monitoring, sMRM—scheduled multiple reaction monitoring, SP—specificity, SM—sphingomyelin, TAG—triacylglycerol, TMS—trimethylsilyl, TOF—time-of-flight, TRF—transferrin, UHPLC—ultra-high performance liquid chromatography, WHO—World Health Organization.

### Definitions

In order that the present disclosure may be more readily understood, certain terms are first defined. Additional definitions are set forth throughout the present description.

As used herein, “ovarian cancer” is a malignant tumor of the ovary. There are several types of ovarian cancers such as malignant serous tumors (low or high grade), mucinous tumors, endometrioid tumors, clear cell tumors, transitional cell tumors, epithelial-stromal tumors, adenosarcomas, carcinosarcomas, granulosa tumors, Sertoli-Leydig tumors, germ cell tumors such as teratomas and mixed germ cell tumors, unclassified tumors, metastatic tumors from nonovarian origin and also tumors with borderline malignancy.

The terms “subject,” “host,” “patient,” and “individual” are used interchangeably herein to refer to any mammalian subject for whom diagnosis or therapy is desired, particularly humans. The subject may have previously suffered from ovarian cancer or the subject may be a healthy individual with no previous signs or symptoms of ovarian cancer. The subject may be a premenopausal or postmenopausal individual.

As used in this description, a “small molecule biomarker” relates to the small molecule biomarkers shown in Table 2. In some embodiments, a “small molecule biomarker” relates to the small molecule biomarkers shown in Table 3 or 4.

As used herein, a “small molecule biomarker from Group A” and a “small molecule biomarker from Group B” relate to the small molecule groups shown in Table 1.

As used herein, a “lipid biomarker” relates to the lipid biomarkers shown in Table 4.

As used herein, a “protein biomarker” relate to any protein used as a biomarker for ovarian cancer, such as cancer antigen 125 (CA-125), human epididymal protein-4 (HE4), prealbumin, apolipoprotein A-1 (ApoA1), beta-2-microglobulin, transferrin (TRF), follicle-stimulating hormone (FSH) and C-reactive protein (CRP).

As used herein, a “sample” is a biological sample obtained from a subject or a group or population of subjects. The sample may be a blood sample, a serum sample, a plasma sample, a saliva sample, an urine sample or a fraction thereof. Blood serum and plasma samples are typical. The sample can be prepared with techniques well known in the art. In certain embodiments, the blood sample is a blood spot dried on a filter. Alternatively, both the sample from the subject and the control sample may also be tissue samples, e.g., ovarian tissue sample, or an ovarian cyst fluid sample.

As used herein, a “control” may be a control sample. A control may also be a concentration determined from a sample from a single healthy individual or a subject with benign tumor or other medical condition causing similar symptoms to ovarian cancer, or the same subject before developing malignant tissue. The control may also be a sample that represents a combination of samples from a generalized population of healthy individuals. Alternatively, the control may be a control value or a set of data concerning the biomarker in a sample previously determined, calculated or extrapolated, or may have yet to be determined, calculated or extrapolated, or may also be taken from the literature.

A control as used herein, i.e., a control value or a control sample, is typically representative of a group of subjects or a population of subjects. In this context, “representative” means that the biomarker concentration(s) reflected by said control value to which a comparison is made in the context of the present disclosure correspond(s) to the average concentration value(s) of said biomarker concentration(s) in corresponding individual samples from the subjects of said group or population. Likewise, in the case of a control sample “representative” means that the biomarker concentration(s) in said control sample to which a comparison is made in the context of the present disclosure correspond(s) to the average concentration(s) of said biomarker concentration(s) in corresponding individual samples from the subjects of said group or population. Typically, the concentrations of all biomarker concentrations in said control sample correspond to the average concentrations of said biomarker concentrations in corresponding individual samples from the subjects of said group or population. An individual with such values can be considered a “healthy individual” for the purposes of the present disclosure.

A control sample can be particularly suitably compared to the subject's sample if it has been obtained from the same type of biological tissue or source in the same, or essentially the same, manner. For example, if the subject's sample is a serum sample or a plasma sample, a corresponding control sample will likewise be a serum sample or a plasma sample, respectively.

It will be appreciated that a useful control value for the purposes of the present disclosure is typically one that has been, or is, obtained using any one of the suitable control samples described herein.

As used herein, in the small molecule nomenclature X:Y, X indicates the number of total carbon atoms in the fatty acid(s) (FA) portions of the molecule, and Y the total number of double bonds in the fatty acid portion(s) of the molecule.

The nomenclature NB indicates, for a DAG molecule, A and B types of fatty acid moieties attached to the glycerol backbone of the molecule. The fatty acid moieties A and B can be attached to any of the two bonding positions of the glycerol backbone of the molecule.

The nomenclature (dC/A) indicates, for a molecule of Cer, Gb3, Glc/GalCer and LacCer, C the type of long-chain base with an amide-linked, and A, fatty acid moiety.

The nomenclature A/B/C indicates A, B and C types of fatty acid moieties attached to the glycerol backbone of the molecule. The fatty acid moieties A, B and C can be attached to any of the three bonding positions of the glycerol backbone of the molecule.

The nomenclature sn1 and sn2 indicate the sn1 and sn2 positions of the glycerol backbone, respectively, to which the fatty acid moiety is attached.

A “treatment” and “therapy” are used interchangeably in the present description and may comprise any therapeutic treatment or operations typically given to a subject having ovarian cancer, such as, but not limited to, surgery, chemotherapy, radiation therapy, hormonal therapy, anti-angiogenic therapy, therapies targeting homologous recombination deficiency, antibody therapy or other targeted therapy utilizing ovarian cancer specific signalling pathways. The treatment may comprise treatment having a direct effect on the metabolism of the malignant tissue. Ovarian cancer therapy may comprise administering a pharmaceutical agent affecting lipid metabolism.

The term “effectiveness of a treatment” and “effectiveness of a therapy” are taken to mean the ability of a treatment and therapy to achieve the therapeutic purpose for which it is administered.

A “pharmaceutical”, “drug”, “medicament” and “medicine” are used interchangeably in the present description and may comprise any pharmaceutical typically given to a subject having ovarian cancer.

As used herein, a “composition” and “kit” are used for diagnosing, predicting and detecting ovarian cancer and comprise means and elements for assaying the small molecule biomarkers described in the present disclosure.

As used herein, a “preparation” is used in the assays determining the small molecule biomarkers described in the present disclosure for diagnosing, predicting and detecting ovarian cancer.

For the purposes of the present disclosure, the terms “obtaining data”, “collecting data”, “obtaining information” and “collecting information” may be used interchangeably.

The terms “the disclosure, description or invention”, “in accordance with the disclosure, description or invention”, “according to the disclosure, description or invention”, “the present disclosure, description or invention” as used herein, are intended to refer to all aspects and embodiments of the disclosure described and/or claimed herein.

As used herein, the term “comprising” is to be construed as encompassing both “including” and “consisting of”.

As used herein “determining” in reference to a molecular biomarker or a protein biomarker as disclosed herein refers to quantitatively or relatively determining an amount of a biomarker in a sample. For quantitative determination, either the absolute or precise amount of the biomarker in a sample is determined. The relative amount or level of a biomarker in a sample, may alternatively be determined, e.g., the biomarker amount in the sample is determined to be enlarged or diminished with respect to a control as described herein.

### EXAMPLES

The following examples are provided to illustrate various aspects of the present disclosure. They are not intended to limit the disclosure, which is defined by the accompanying claims.

### Example 1. Materials and Methods

### Description of Study Cohorts and Samples

Analyses in two study cohorts led to the current disclosure, referred here as cohort I and II. Cohort II had more early-stage ovarian cancers than cohort I. Cohort I had 100 subjects without malignant disease (control group) and 158 ovarian cancer patients. Cohort II had 109 subjects without malignant disease (control group) and 62 ovarian cancer patients. The serum samples of both these cohorts were collected from preoperative primary ovarian cancer patients as well as from patients without ovarian cancer at the Charité Medical University (Berlin, Germany). The Ethics Committee approved the use of the samples for the study. The patient's informed consent was obtained prior to surgery or during subsequent treatment, sample collection and documentation of clinical and surgical data. The study population without ovarian cancer consisted of a group of patients with benign tumors, endometriosis, cysts, uterus myomatosus and other conditions causing similar symptoms to ovarian cancer. Blood was collected within the Tumor Bank Ovarian Cancer project (http://toc-network.de) using serum tubes containing clot activators (Vacutainer, BD, Medical-Pharmaceutical System, Franklin Lakes, N.J.). Collected blood was clotted for 30 min to 2 h at room temperature and serum was separated by centrifugation at 1200 g for 15 minutes. Serum was aliquoted and stored at −80° C.

### GC×GC-TOF Method

For the serum samples of cohort I, 400 μl methanol and 10 μl standard mixture (valine-d8 (37.6 mg/I), heptadecanoic acid (186.5 mg/I), succinic acid-d4 (62.9 mg/I), glutamic acid-d5 (103.5 mg/I)) was added to 30 μl of the sample. The samples were vortexed for 2 min. After 30 minutes at room temperature the samples were centrifuged for 5 min at 10000 rpm. 200 μl of the supernatant was moved to a GC vial and evaporated to dryness under nitrogen. The samples were derivatized with 25 μl methoxyamine (45° C., 60 minutes) and 25 μl Nmethyltrimethylsilyltrifluoroacetamide (45° C., 60 minutes) and 50 μl of hexane with retention index compounds and injection standard (4,4′-dibromooctafluorobiphenyl) was added to samples.

For the analysis, a Leco Pegasus 4D GC×GC-TOFMS instrument (Leco Corp., St. Joseph, Mich.) equipped with a cryogenic modulator was used. The GC part of the instrument was an Agilent 6890 gas chromatograph (Agilent Technologies, Palo Alto, Calif.), equipped with split/splitless injector. The first-dimension chromatographic column was a 10-m Rxi-5MS capillary column with an internal diameter of 0.18 mm and a stationary-phase film thickness of 0.18 μm, and the second-dimension chromatographic column was a 1.5 m BPX-50 capillary column with an internal diameter of 100 μm and a film thickness of 0.1 μm. A methyl deactivated retention gap (1.5 m×0.53 mm i.d.) was used in the front of the first column. High-purity helium was used as the carrier gas at a constant pressure mode (40 psig). A 4-s separation time was used in the second dimension. The MS spectra were measured at 45-700 atomic mass unit (amu) with 100 spectra/second. For the injection, a splitless injection (1.0 μl) at 240° C. was utilized. The temperature program was as follows: the first-dimension column oven ramp began at 50° C. with a 2 minute hold after which the temperature was programmed to 240° C. at a rate of 7° C./minute and further to 300° C. at a rate of 25° C./minute and then held at this temperature for 3 minute. The second-dimension column temperature was maintained 15° C. higher than the corresponding first-dimension column. The programming rate and hold times were the same for the two columns.

ChromaTOF vendor software (LECO) was used for within-sample data processing, and inhouse made software Guineu (Castillo et al., 2011, Anal Chem) was used for alignment, normalization and peak matching across samples. The peaks were first filtered based on number of detected peaks in the total profile of all sample runs. The normalization for uncalibrated metabolites was performed by correction for internal standard C17:0. 27 of the metabolites were checked manually in each serum sample for correct integration and identification. Other mass spectra from the GC×GC-TOFMS analysis were searched against National Institutes of Standards and Technology 05 (NIST05) mass spectral library.

### GC-MS Method

For the serum of cohort II, a modified method of the GC×GC-TOF method was used for sample pretreatment and instrument parameters. After samples were thawed unassisted on top of ice, 20 μl of serum was dispensed into test tube and extraction solution (600 μl) with internal standard (succinic acid-d4, 0.3 μg/ml in methanol) was added to the sample. The sample was mixed and left in the freezer for 10 minutes (−20° C.). Samples were centrifuged at 14,000 rpm for 10 minutes at 4° C., and the supernatants (300 μl) were evaporated to dryness under nitrogen. Analytes were converted into their trimethylsilyl (TMS) derivatives by adding 50 μl N,O-bis(trimethylsilyl)trifluoroacetamide (BSTFA) to the sample and the mixture was heated at 50° C. for 30 minutes. After cooling samples to the room temperature, 50 μl of hexane was added and mixed with Vortex mixer for 10 seconds. The quantification was based on internal standard method.

The instrument was Agilent Technologies GC-MS system (Agilent Technologies; Palo Alto, Calif.). The system consisted of a 7683 autosampler and a 6890N gas chromatograph coupled to a 5973N mass spectrometry. Chromatographic conditions were as follows: VF-5 ms capillary column (30 m×0.25 mm, film 0.25 μm) with a built-in guard column (EZ-Guard, 10 m) (Agilent Technologies; Palo Alto, Calif., USA) was used with pulsed splitless injection. Injection port temperature was 250° C. and injection volume was 1 μl. The oven temperature was held at 60° C. for 2 minutes, then increased to 170° C. at 10° C. per minute rate, and then increased to 300° C. at 20° C. per minute rate, and held at 300° C. for 6.5 minutes. The carrier gas was helium with constant flow of 32 cm/sec (equal to 1 ml/min). The temperatures of the MSD transfer line heater, ionization source and quadrupole were maintained at 250° C., 230° C., and 150° C., respectively. The mass spectrometer was operated in electron ionization (EI) mode with the electron energy 70 eV. The total measurement time was 26 min. A solvent delay of 8 minutes was applied. For GC/EI-MS in the selected ion monitoring (SIM) mode was used to record target and qualifier ions measurements. Dwell time was 25 milliseconds (ms) for all recorded ions.

### LC-MS/MS Lipidomics Analysis

Lipidomic analyses of cohorts I and II were performed using two platforms, a global screening method and a phosphosphingolipid platform. Lipids for the screening method were extracted using a modified Folch extraction (Folch et al., 1957, J. Biol Chem) and protein precipitation in methanol was used for the extraction of phosphosphingolipids. Prior to extraction, samples were thawed at +4° C., and Hamilton MICROLAB STAR system (Hamilton Robotics, Switzerland) was used for the extraction. For the screening method, samples (10 μl) were aliquoted into a 96-well plate, and internal standard mixture (20 μL) containing a known amount of synthetic internal standards was added followed by chloroform (450 μl). Organic phase separation was facilitated by adding 20 mM acetic acid and centrifuging the plate for 5 minutes at 500×g. The lower organic phase (360 μl) was transferred into a new 96-well plate. The remaining water-containing phase was washed with additional chloroform (360 μl) followed by centrifugation and removal of the remaining organic phase. The two organic phases were pooled and evaporated under N₂ until dryness. The lipid extracts were then re-dissolved in chloroform:methanol (1:2, v/v). For the analysis of phosphosphingolipids, samples (25 μl) were aliquoted into a 96-well plate, and ice-cold methanol containing 0.1% butylated hydroxytoluene (500 μL) was added to each sample, followed by internal standard mixture (25 μL) containing a known amount of synthetic standards. Samples were mixed and incubated for 10 minutes. After centrifugation, supernatant (450 μl) was transferred into a new 96-well plate, evaporated under N₂ until dryness and re-dissolved in methanol (200 μL).

Lipidomics screening and phosphosphingolipid platforms were both analyzed on a hybrid triple quadrupole/linear ion trap mass spectrometer (QTRAP 5500, AB Sciex, Concors, Canada) equipped with an ultra-high performance liquid chromatography (UHPLC) (Nexera-X2, Shimadzu). Chromatographic separation of the lipidomics screening platform was performed on Acquity BEH C18, 2.1×50 mm id. 1.7 μm column (Waters, Mass., USA). Mobile phases consisted of (A) 10 mM ammonium acetate in LC-MS grade water with 0.1% formic acid, and (B) 10 mM ammonium acetate in acetonitrile:2-propanol (3:4, VN) with 0.1% formic acid (FA). Chromatographic separation of phosphosphingolipid platform was performed on AQUASIL C18, 2.1×50 mm, 5 μm (Thermo Fisher, Massachusetts, USA), column set at 60° C. Mobile phases consisted of (A) 10 mM ammonium acetate in LC-MS grade water with 0.1% formic acid, and (B) 10 mM ammonium acetate in methanol:2-Propanol (1:2) with 0.1% formic acid.

For the MS analysis, a targeted approach in positive ion mode was used for both platforms. Data was collected using scheduled multiple reaction monitoring (sMRM™) algorithm for the lipidomics screening platform (Weir et al., 2013, J Lipid Res) and multiple reaction monitoring (MRM) for phosphosphingolipids. Mass spectrometer parameters were optimized based on lipid class. Lipidomics data were processed using Analyst and MultiQuant 3.0 software (QTRAP 5500, AB Sciex, Concors, Canada), area or height ratios of analyte and its corresponding internal standard (IS) peak were normalized with IS amount and sample volume.

### Statistical Analyses

All statistical analyses were performed using R, version x64 3.3.2. For the two-group comparisons unpaired t-tests were performed after log 2 transformation of the data. In addition, mean relative differences were calculated between the patients with malignant tumors and control group. Association of small molecule biomarkers to overall survival was analysed by cox proportional hazards regression test. AUC values were calculated using R package ROCR. Sensitivity and specificity values were calculated with a cut-off value where the sum of sensitivity and specificity was at maximum. Predictive models combining small molecule biomarker ratios and CA-125 were binary logistic regression models.

### Example 2. Results

### Increased and Decreased Small Molecule Biomarkers

Table 2 shows statistically significantly (p<0.05) increased or decreased small molecule biomarkers in ovarian cancer patients as compared to control group. The results were derived from cohort I, except for acetoacetic acid, where its concentration was determined by more accurate method in cohort II.

TABLE 2
Increased (group A) (a) and decreased (group B) (b) small molecule biomarkers in ovarian cancer
patients. Abbreviations are described in the detailed description of the present disclosure.

| | CHANGE (%) | P-VALUE | CLASS |
|---|---|---|---|
| Table 2a. Group A molecules | | | |
| Cysteine | 27.3 | 4.9E−02 | Amino acids I |
| Glutamic acid | 25.7 | 1.6E−03 | Amino acids I |
| Glutamine | 10.6 | 2.2E−02 | Amino acids I |
| Glycine | 56.1 | 4.1E−04 | Amino acids I |
| 2,3-Dihydroxybutyric acid | 29.3 | 5.8E−04 | Butyric acids |
| 2,4-Dihydroxybutyric acid | 29.8 | 3.2E−04 | Butyric acids |
| 2-Aminobutyric acid | 99.7 | 2.1E−04 | Butyric acids |
| 2-Hydroxybutyric acid | 48.8 | 1.5E−09 | Butyric acids |
| 3,4-Dihydroxybutyric acid | 90.7 | 1.8E−25 | Butyric acids |
| 3-Hydroxybutyric acid | 196.6 | 4.7E−20 | Butyric acids |
| Cer(d16:1/18:0) | 24.6 | 1.3E−03 | Ceramides with FA 16:0, 18:0, 20:0, 22:0, 24:1 |
| Cer(d18:0/16:0) | 27.0 | 3.2E−03 | Ceramides with FA 16:0, 18:0, 20:0, 22:0, 24:1 |
| Cer(d18:0/18:0) | 49.7 | 5.7E−05 | Ceramides with FA 16:0, 18:0, 20:0, 22:0, 24:1 |
| Cer(d18:0/20:0) | 22.0 | 1.8E−02 | Ceramides with FA 16:0, 18:0, 20:0, 22:0, 24:1 |
| Cer(d18:0/24:1) | 16.4 | 1.5E−02 | Ceramides with FA 16:0, 18:0, 20:0, 22:0, 24:1 |
| Cer(d18:1/16:0) | 26.7 | 1.6E−05 | Ceramides with FA 16:0, 18:0, 20:0, 22:0, 24:1 |
| Cer(d18:1/18:0) | 71.4 | 7.2E−12 | Ceramides with FA 16:0, 18:0, 20:0, 22:0, 24:1 |
| Cer(d18:1/20:0) | 39.7 | 9.9E−08 | Ceramides with FA 16:0, 18:0, 20:0, 22:0, 24:1 |
| Cer(d18:1/22:0) | 11.9 | 2.1E−02 | Ceramides with FA 16:0, 18:0, 20:0, 22:0, 24:1 |
| Cer(d18:1/24:1) | 30.5 | 1.1E−06 | Ceramides with FA 16:0, 18:0, 20:0, 22:0, 24:1 |
| Cer(d18:2/16:0) | 15.2 | 1.2E−02 | Ceramides with FA 16:0, 18:0, 20:0, 22:0, 24:1 |
| Cer(d18:2/18:0) | 26.2 | 5.2E−04 | Ceramides with FA 16:0, 18:0, 20:0, 22:0, 24:1 |
| Cer(d18:2/24:1) | 13.5 | 1.5E−02 | Ceramides with FA 16:0, 18:0, 20:0, 22:0, 24:1 |
| Cer(d20:1/22:0) | 21.6 | 2.8E−03 | Ceramides with FA 16:0, 18:0, 20:0, 22:0, 24:1 |
| Cer(d20:1/24:1) | 43.9 | 2.1E−07 | Ceramides with FA 16:0, 18:0, 20:0, 22:0, 24:1 |
| Eicosenoic acid (C20:1) | 11.6 | 2.1E−02 | Fatty acids |
| Lauric acid (C12:0) | 14.9 | 4.1E−02 | Fatty acids |
| Linolenic acid (C18:3) | 37.3 | 2.3E−05 | Fatty acids |
| Myristic acid (C14:0) | 20.8 | 5.7E−03 | Fatty acids |
| Oleic acid (C18:1) | 16.7 | 6.0E−04 | Fatty acids |
| Palmitelaidic acid (C16:1) | 27.6 | 2.9E−04 | Fatty acids |
| Palmitic acid (C16:0) | 8.0 | 1.4E−02 | Fatty acids |
| LPC 16:0_sn1 | 40.7 | 6.3E−03 | LPL I |
| LPC 20:1_sn2 | 20.6 | 3.8E−02 | LPL I |
| LPC 20:5_sn1 | 18.6 | 2.6E−02 | LPL I |
| LPC 22:6_sn1 | 20.5 | 3.7E−02 | LPL I |
| LPC O-18:1 | 35.9 | 1.9E−02 | LPL I |
| LPC P-18:1 | 29.2 | 4.4E−02 | LPL I |
| LPE 18:0_sn1 | 35.5 | 1.4E−03 | LPL I |
| LPE 18:0_sn2 | 33.5 | 2.6E−03 | LPL I |
| 3-Hydroxyisovaleric acid | 63.8 | 2.3E−04 | Other I |
| 4-Hydroxyphenyllactic acid | 12.6 | 1.5E−02 | Other I |
| Acetoacetic acid | 271.2 | 1.6E−08 | Other I |
| Adipic acid | 35.9 | 7.6E−07 | Other I |
| Alpha-ketoglutaric acid | 52.1 | 4.8E−02 | Other I |
| Ethanolamine | 49.2 | 3.1E−07 | Other I |
| Glycerol | 64.2 | 9.7E−14 | Other I |
| PE O-36:1 | 62.4 | 1.9E−05 | PE O I |
| PE O-36:2 | 93.3 | 6.5E−08 | PE O I |
| PE O-36:3 | 66.0 | 9.3E−05 | PE O I |
| Arabinose | 52.5 | 1.3E−05 | Sugars |
| Erythritol | 34.3 | 3.4E−05 | Sugars |
| Glucopyranose | 17.0 | 2.6E−02 | Sugars |
| Maltose | 814.4 | 5.0E−23 | Sugars |
| Mannonic acid | 103.7 | 6.0E−14 | Sugars |
| Melibiose | 392.4 | 6.6E−08 | Sugars |
| Myo-Inositol | 32.4 | 4.2E−07 | Sugars |
| Turanose | 434.5 | 2.4E−03 | Sugars |
| Xylitol | 30.1 | 5.0E−09 | Sugars |
| TAG(16:0/18:1/18:1) | 9.2 | 3.7E−02 | TAG I |
| TAG(18:0/18:2/18:2) | 18.1 | 1.2E−02 | TAG I |
| TAG(18:1/18:1/18:1) | 8.2 | 3.9E−02 | TAG I |
| TAG(18:1/18:1/20:4) | 28.4 | 6.7E−06 | TAG I |
| TAG(18:1/18:1/22:6) | 54.9 | 5.4E−08 | TAG I |
| TAG(18:2/18:2/18:2) | 118.1 | 3.3E−07 | TAG I |
| Table 2b. Group B molecules | | | |
| Alanine | −44.7 | 3.0E−19 | Amino acids II |
| Methionine | −25.9 | 5.7E−17 | Amino acids II |
| Phenylalanine | −13.2 | 1.2E−03 | Amino acids II |
| Proline | −21.9 | 3.0E−05 | Amino acids II |
| Serine | −13.1 | 1.5E−03 | Amino acids II |
| Threonine | −36.3 | 1.4E−15 | Amino acids II |
| Tryptophan | −66.8 | 2.1E−21 | Amino acids II |
| Tyrosine | −24.9 | 1.5E−03 | Amino acids II |
| Valine | −8.2 | 1.2E−02 | Amino acids II |
| CE 14:0 | −22.3 | 4.1E−06 | CE |
| CE 14:1 | −22.0 | 4.7E−05 | CE |
| CE 15:0 | −16.8 | 2.0E−04 | CE |
| CE 16:0 | −12.5 | 1.4E−04 | CE |
| CE 16:2 | −21.1 | 2.0E−07 | CE |
| CE 17:0 | −16.5 | 1.2E−04 | CE |
| CE 17:1 | −10.1 | 2.1E−02 | CE |
| CE 18:0 | −26.7 | 4.9E−10 | CE |
| CE 18:1 | −12.7 | 1.9E−04 | CE |
| CE 18:2 | −21.9 | 7.2E−10 | CE |
| CE 18:3 | −23.2 | 3.8E−10 | CE |
| CE 19:1 | −0.9 | 4.6E−02 | CE |
| CE 20:2 | −23.3 | 2.6E−07 | CE |
| CE 20:3 | −21.3 | 5.8E−06 | CE |
| CE 20:4 | −7.4 | 5.0E−02 | CE |
| CE 22:3 | −15.8 | 3.9E−02 | CE |
| CE 22:4 | −14.9 | 1.6E−03 | CE |
| CE 22:5 | −35.3 | 2.0E−08 | CE |
| CE 24:6 | −22.7 | 1.2E−04 | CE |
| Cer(d16:1/23:0) | −24.9 | 5.7E−04 | Ceramides with FA 14:0, 23:0, 24:0, 26:0 |
| Cer(d16:1/24:0) | −29.3 | 6.1E−08 | Ceramides with FA 14:0, 23:0, 24:0, 26:0 |
| Cer(d16:1/26:0) | −17.1 | 4.0E−03 | Ceramides with FA 14:0, 23:0, 24:0, 26:0 |
| Cer(d18:0/23:0) | −17.4 | 3.8E−03 | Ceramides with FA 14:0, 23:0, 24:0, 26:0 |
| Cer(d18:0/24:0) | −14.8 | 2.5E−03 | Ceramides with FA 14:0, 23:0, 24:0, 26:0 |
| Cer(d18:1/14:0) | −10.3 | 4.0E−02 | Ceramides with FA 14:0, 23:0, 24:0, 26:0 |
| Cer(d18:1/24:0) | −11.9 | 8.6E−03 | Ceramides with FA 14:0, 23:0, 24:0, 26:0 |
| Cer(d18:2/23:0) | −18.5 | 5.6E−04 | Ceramides with FA 14:0, 23:0, 24:0, 26:0 |
| Cer(d18:2/24:0) | −22.4 | 1.1E−05 | Ceramides with FA 14:0, 23:0, 24:0, 26:0 |
| Cer(d18:2/26:0) | −14.2 | 4.0E−02 | Ceramides with FA 14:0, 23:0, 24:0, 26:0 |
| DAG(14:0/18:1) | −39.1 | 6.9E−04 | DAG |
| DAG(14:0/18:2) | −38.3 | 1.6E−03 | DAG |
| DAG(18:1/20:3) | −31.3 | 1.1E−02 | DAG |
| DAG(16:0/16:1) | −27.6 | 1.3E−02 | DAG |
| DAG(18:0/20:4) | −22.0 | 3.9E−02 | DAG |
| Gb3(d18:1/16:0) | −14.8 | 3.0E−04 | Gb3 |
| Gb3(d18:1/18:0) | −13.4 | 2.2E−02 | Gb3 |
| Gb3(d18:1/22:0) | −19.5 | 4.5E−04 | Gb3 |
| Gb3(d18:1/23:0) | −16.2 | 5.6E−03 | Gb3 |
| Gb3(d18:1/24:0) | −23.1 | 8.6E−05 | Gb3 |
| Glc/GalCer(d16:1/20:0) | −16.8 | 9.7E−04 | Glc/GalCer |
| Glc/GalCer(d16:1/22:0) | −23.4 | 1.5E−05 | Glc/GalCer |
| Glc/GalCer(d16:1/23:0) | −19.3 | 3.8E−04 | Glc/GalCer |
| Glc/GalCer(d16:1/24:0) | −23.1 | 5.0E−06 | Glc/GalCer |
| Glc/GalCer(d18:1/16:0) | −13.3 | 1.1E−02 | Glc/GalCer |
| Glc/GalCer(d18:1/18:0) | −16.1 | 7.5E−04 | Glc/GalCer |
| Glc/GalCer(d18:1/20:0) | −19.7 | 9.2E−05 | Glc/GalCer |
| Glc/GalCer(d18:1/22:0) | −25.9 | 2.6E−07 | Glc/GalCer |
| Glc/GalCer(d18:1/23:0) | −23.8 | 7.8E−07 | Glc/GalCer |
| Glc/GalCer(d18:1/24:0) | −24.2 | 4.7E−07 | Glc/GalCer |
| Glc/GalCer(d18:1/26:0) | −21.0 | 9.4E−05 | Glc/GalCer |
| Glc/GalCer(d18:2/20:0) | −23.3 | 1.7E−04 | Glc/GalCer |
| Glc/GalCer(d18:2/22:0) | −17.9 | 5.0E−04 | Glc/GalCer |
| Glc/GalCer(d18:2/23:0) | −21.6 | 5.2E−05 | Glc/GalCer |
| Glc/GalCer(d18:2/24:0) | −23.9 | 4.8E−08 | Glc/GalCer |
| LacCer(d16:1/16:0) | −21.1 | 5.3E−05 | LacCer |
| LacCer(d18:1/16:0) | −23.5 | 2.0E−08 | LacCer |
| LacCer(d18:1/22:0) | −27.7 | 7.6E−10 | LacCer |
| LacCer(d18:1/23:0) | −26.0 | 1.7E−07 | LacCer |
| LacCer(d18:1/24:0) | −23.2 | 1.5E−08 | LacCer |
| LacCer(d18:1/24:1) | −21.0 | 2.8E−06 | LacCer |
| LacCer(d18:2/16:0) | −25.2 | 5.5E−08 | LacCer |
| LacCer(d18:2/22:0) | −18.2 | 3.9E−02 | LacCer |
| LacCer(d18:2/24:0) | −19.7 | 4.0E−03 | LacCer |
| LacCer(d18:2/24:1) | −10.7 | 2.6E−02 | LacCer |
| LPC 14:0_sn1 | −19.1 | 1.6E−03 | LPL II |
| LPC 14:0_sn2 | −30.3 | 2.3E−05 | LPL II |
| LPC 18:2_sn1 | −29.7 | 1.9E−07 | LPL II |
| LPC 18:2_sn2 | −34.7 | 2.3E−12 | LPL II |
| LPC 20:0_sn1 | −16.6 | 3.4E−03 | LPL II |
| LPC 20:0_sn2 | −14.6 | 1.1E−02 | LPL II |
| LPC 20:2_sn1 | −5.7 | 3.7E−02 | LPL II |
| LPC 20:2_sn2 | −16.4 | 3.2E−05 | LPL II |
| LPC 20:3_sn1 | −16.1 | 2.3E−03 | LPL II |
| LPC 20:3_sn2 | −24.4 | 1.4E−06 | LPL II |
| LPC 22:0_sn1 | −28.7 | 6.3E−10 | LPL II |
| LPC 22:4_sn1 | −11.1 | 1.7E−02 | LPL II |
| LPC 22:4_sn2 | −19.6 | 4.9E−05 | LPL II |
| LPC 24:0_sn1 | −28.7 | 1.5E−12 | LPL II |
| LPC 24:0_sn2 | −28.9 | 2.3E−13 | LPL II |
| LPC 0-20:0 | −15.5 | 7.1E−04 | LPL II |
| LPC 0-22:0 | −25.3 | 4.8E−11 | LPL II |
| LPC 0-22:1 | −17.5 | 1.9E−03 | LPL II |
| LPC 0-24:0 | −26.6 | 2.9E−10 | LPL II |
| LPC 0-24:1 | −17.8 | 8.2E−05 | LPL II |
| LPC 0-24:2 | −33.0 | 1.2E−08 | LPL II |
| LPE 18:2_sn1 | −40.1 | 1.5E−11 | LPL II |
| LPE 18:2_sn2 | −35.4 | 4.6E−10 | LPL II |
| LPE 20:4_sn1 | −16.4 | 2.0E−03 | LPL II |
| 2-oxo-3-methylpentanoic acid | −26.9 | 7.0E−09 | Other II |
| 2-Oxoisovaleric acid | −18.5 | 2.3E−02 | Other II |
| Cholesterol | −31.3 | 1.8E−06 | Other II |
| Glyceric acid | −28.1 | 6.5E−04 | Other II |
| Glycerol-3-phosphate | −21.4 | 8.2E−03 | Other II |
| lndole-3-acetic acid | −27.2 | 3.0E−03 | Other II |
| Ketoleucine | −45.2 | 7.0E−19 | Other II |
| Lactic acid | −29.0 | 3.2E−11 | Other II |
| Malic acid | −28.5 | 2.1E−06 | Other II |
| Pyruvic acid | −32.0 | 3.2E−02 | Other II |
| PC 28:0 | −59.0 | 1.5E−15 | PC |
| PC 30:0 | −43.4 | 4.0E−15 | PC |
| PC 30:1 | −52.1 | 2.4E−13 | PC |
| PC 30:2 | −61.4 | 2.1E−14 | PC |
| PC 31:0 | −26.0 | 1.6E−07 | PC |
| PC 31:1 | −36.9 | 1.9E−08 | PC |
| PC 32:0 | −15.5 | 4.3E−05 | PC |
| PC 32:1 | −29.0 | 1.9E−06 | PC |
| PC 32:2 | −52.5 | 4.0E−19 | PC |
| PC 32:3 | −58.5 | 4.2E−17 | PC |
| PC 33:0 | −17.1 | 2.0E−03 | PC |
| PC 33:1 | −25.2 | 5.2E−06 | PC |
| PC 33:2 | −35.6 | 6.9E−13 | PC |
| PC 33:3 | −42.0 | 4.2E−11 | PC |
| PC 34:0 | −11.9 | 3.6E−03 | PC |
| PC 34:1 | −10.2 | 1.9E−02 | PC |
| PC 34:2 | −28.6 | 4.5E−10 | PC |
| PC 34:3 | −37.9 | 9.7E−14 | PC |
| PC 34:4 | −52.1 | 5.6E−16 | PC |
| PC 34:5 | −41.3 | 1.4E−05 | PC |
| PC 35:0 | −21.3 | 5.5E−04 | PC |
| PC 35:1 | −15.5 | 4.0E−03 | PC |
| PC 35:2 | −26.3 | 2.6E−06 | PC |
| PC 35:3 | −39.8 | 3.1E−15 | PC |
| PC 35:4 | −25.8 | 1.5E−07 | PC |
| PC 36:0 | −21.7 | 1.3E−07 | PC |
| PC 36:2 | −35.1 | 2.0E−13 | PC |
| PC 36:3 | −36.0 | 2.2E−14 | PC |
| PC 36:5 | −32.1 | 1.4E−07 | PC |
| PC 36:6 | −46.4 | 2.0E−09 | PC |
| PC 36:7 | −27.4 | 2.1E−05 | PC |
| PC 37:1 | −20.5 | 4.6E−06 | PC |
| PC 37:2 | −36.3 | 6.0E−13 | PC |
| PC 37:3 | −30.4 | 7.4E−09 | PC |
| PC 37:6 | −20.3 | 8.9E−03 | PC |
| PC 38:0 | −29.1 | 5.0E−11 | PC |
| PC 38:1 | −9.8 | 2.8E−02 | PC |
| PC 38:2 | −27.9 | 3.7E−08 | PC |
| PC 38:3 | −30.6 | 1.5E−09 | PC |
| PC 38:4 | −24.1 | 6.4E−06 | PC |
| PC 38:5 | −15.7 | 6.6E−05 | PC |
| PC 38:6 | −35.0 | 2.6E−10 | PC |
| PC 38:7 | −30.9 | 7.8E−05 | PC |
| PC 39:0 | −18.7 | 9.9E−07 | PC |
| PC 39:4 | −19.5 | 5.6E−06 | PC |
| PC 39:5 | −12.0 | 3.5E−02 | PC |
| PC 40:1 | −22.9 | 3.6E−08 | PC |
| PC 40:3 | −15.8 | 1.6E−04 | PC |
| PC 40:4 | −29.9 | 9.0E−12 | PC |
| PC 40:5 | −14.8 | 1.4E−03 | PC |
| PC 40:8 | −33.4 | 6.8E−10 | PC |
| PC O-32:0 | −24.5 | 2.7E−08 | PC O/P |
| PC O-32:1 | −31.3 | 7.1E−10 | PC O/P |
| PC O-34:0 | −20.9 | 1.1E−06 | PC O/P |
| PC O-34:1 | −22.2 | 1.2E−08 | PC O/P |
| PC O-34:2 | −39.7 | 6.7E−17 | PC O/P |
| PC O-36:1 | −27.6 | 1.5E−07 | PC O/P |
| PC O-36:2 | −36.3 | 4.2E−14 | PC O/P |
| PC O-36:3 | −38.7 | 4.8E−16 | PC O/P |
| PC O-36:4 | −23.1 | 2.4E−09 | PC O/P |
| PC O-38:0 | −21.3 | 8.0E−09 | PC O/P |
| PC O-38:1 | −27.9 | 5.8E−12 | PC O/P |
| PC O-38:2 | −28.3 | 4.8E−09 | PC O/P |
| PC O-38:3 | −31.7 | 2.9E−11 | PC O/P |
| PC O-38:4 | −31.5 | 7.2E−12 | PC O/P |
| PC O-38:5 | −16.0 | 3.4E−05 | PC O/P |
| PC O-38:6 | −13.5 | 7.2E−03 | PC O/P |
| PC O-40:1 | −23.6 | 4.1E−06 | PC O/P |
| PC O-40:3 | −37.5 | 2.5E−10 | PC O/P |
| PC O-40:4 | −18.1 | 8.5E−04 | PC O/P |
| PC O-40:5 | −20.3 | 1.3E−04 | PC O/P |
| PC P-36:2 | −26.8 | 6.2E−08 | PC O/P |
| PC P-32:0 | −20.2 | 8.1E−07 | PC O/P |
| PC P-32:1 | −21.4 | 1.6E−05 | PC O/P |
| PC P-34:1 | −13.9 | 3.6E−04 | PC O/P |
| PC P-34:2 | −33.3 | 2.7E−13 | PC O/P |
| PC P-36:3 | −17.8 | 2.4E−06 | PC O/P |
| PC P-36:4 | −20.0 | 8.1E−07 | PC O/P |
| PC P-38:1 | −16.5 | 4.7E−04 | PC O/P |
| PC P-38:2 | −17.0 | 2.7E−03 | PC O/P |
| PC P-38:4 | −21.4 | 2.1E−05 | PC O/P |
| PC P-38:5 | −11.3 | 1.5E−03 | PC O/P |
| PC P-38:6 | −22.9 | 5.9E−04 | PC O/P |
| PC P-40:1 | −15.9 | 5.1E−04 | PC O/P |
| PC P-40:2 | −21.6 | 1.3E−05 | PC O/P |
| PC P-40:3 | −23.1 | 3.4E−07 | PC O/P |
| PC P-40:4 | −21.8 | 2.8E−07 | PC O/P |
| PC P-40:5 | −17.3 | 2.4E−05 | PC O/P |
| PC P-40:6 | −6.2 | 3.7E−02 | PC O/P |
| PE 32:1 | −39.1 | 2.3E−04 | PE |
| PE 34:2 | −39.2 | 1.0E−06 | PE |
| PE 34:3 | −49.7 | 8.1E−07 | PE |
| PE 36:2 | −37.9 | 1.5E−06 | PE |
| PE 36:3 | −53.8 | 2.0E−07 | PE |
| PE 36:4 | −30.7 | 2.9E−04 | PE |
| PE 36:5 | −26.5 | 2.3E−02 | PE |
| PE 38:1 | −37.4 | 7.9E−05 | PE |
| PE 38:2 | −29.4 | 2.0E−02 | PE |
| PE 38:3 | −40.4 | 6.8E−07 | PE |
| PE 38:4 | −47.6 | 7.3E−07 | PE |
| PE 38:5 | −33.5 | 5.1E−06 | PE |
| PE 40:4 | −34.8 | 1.2E−03 | PE |
| PE 40:5 | −21.8 | 8.3E−04 | PE |
| PE 40:7 | −42.4 | 1.3E−03 | PE |
| PE O-34:1 | −12.4 | 4.7E−02 | PE 0 II |
| PE O-36:4 | −49.1 | 4.9E−11 | PE 0 II |
| PE O-38:4 | −48.9 | 1.5E−12 | PE 0 II |
| PE O-38:5 | −38.5 | 1.3E−09 | PE 0 II |
| PE O-38:6 | −16.2 | 4.4E−02 | PE 0 II |
| PE P-34:2 | −20.6 | 1.4E−03 | PE P |
| PE P-36:2 | −41.7 | 7.0E−10 | PE P |
| PE P-36:4 | −31.6 | 1.0E−06 | PE P |
| PE P-38:4 | −36.4 | 7.4E−10 | PE P |
| PE P-38:6 | −31.2 | 1.4E−07 | PE P |
| PE P-40:6 | −20.6 | 1.6E−03 | PE P |
| PG 34:1 | −22.9 | 1.1E−02 | PG |
| PG 36:2 | −25.6 | 3.5E−04 | PG |
| PI 32:0 | −49.6 | 3.0E−04 | PI |
| PI 34:0 | −45.7 | 5.7E−04 | PI |
| PI 34:1 | −31.3 | 4.7E−04 | PI |
| PI 34:2 | −31.3 | 6.4E−06 | PI |
| PI 36:1 | −40.1 | 3.8E−07 | PI |
| PI 36:2 | −27.9 | 5.2E−06 | PI |
| PI 36:3 | −51.5 | 9.9E−10 | PI |
| PI 36:4 | −24.3 | 2.7E−03 | PI |
| PI 38:2 | −40.5 | 3.5E−07 | PI |
| PI 38:3 | −33.8 | 1.6E−09 | PI |
| PI 38:5 | −24.2 | 4.3E−05 | PI |
| PI 40:4 | −19.9 | 1.1E−03 | PI |
| S1P d16:1 | −20.0 | 1.4E−08 | S1P/SA1P |
| S1P d18:1 | −19.3 | 1.3E−07 | S1P/SA1P |
| S1P d18:2 | −29.3 | 1.2E−13 | S1P/SA1P |
| SA1P d18:0 | −24.8 | 5.3E−13 | S1P/SA1P |
| SM 30:2 | −35.8 | 2.2E−11 | SM |
| SM 31:0 | −12.2 | 4.2E−02 | SM |
| SM 31:1 | −24.7 | 1.2E−07 | SM |
| SM 31:2 | −12.3 | 7.5E−03 | SM |
| SM 32:0 | −24.2 | 8.6E−08 | SM |
| SM 32:1 | −17.3 | 7.4E−06 | SM |
| SM 32:2 | −28.0 | 3.3E−11 | SM |
| SM 33:1 | −11.6 | 4.7E−03 | SM |
| SM 33:2 | −9.2 | 4.4E−02 | SM |
| SM 34:0 | −15.1 | 5.0E−04 | SM |
| SM 34:1 | −13.2 | 4.6E−04 | SM |
| SM 34:2 | −15.8 | 2.3E−05 | SM |
| SM 35:0 | −25.3 | 3.2E−06 | SM |
| SM 35:1 | −10.3 | 9.7E−03 | SM |
| SM 35:2 | −9.3 | 9.1E−03 | SM |
| SM 36:3 | −14.6 | 2.7E−03 | SM |
| SM 37:0 | −10.1 | 1.6E−02 | SM |
| SM 37:1 | −14.8 | 1.9E−04 | SM |
| SM 37:2 | −34.8 | 1.4E−13 | SM |
| SM 38:0 | −22.5 | 2.0E−05 | SM |
| SM 38:1 | −20.4 | 1.0E−06 | SM |
| SM 38:2 | −19.1 | 2.8E−06 | SM |
| SM 38:3 | −20.2 | 8.5E−05 | SM |
| SM 39:0 | −18.0 | 7.1E−03 | SM |
| SM 39:1 | −27.5 | 1.3E−10 | SM |
| SM 39:2 | −21.6 | 1.5E−03 | SM |
| SM 40:0 | −27.0 | 5.9E−07 | SM |
| SM 40:1 | −23.7 | 4.2E−08 | SM |
| SM 40:2 | −27.3 | 4.5E−12 | SM |
| SM 40:3 | −19.8 | 2.6E−06 | SM |
| SM 41:0 | −32.5 | 1.3E−09 | SM |
| SM 41:1 | −25.2 | 8.0E−12 | SM |
| SM 41:2 | −17.0 | 7.1E−05 | SM |
| SM 42:0 | −30.9 | 3.8E−10 | SM |
| SM 42:1 | −31.1 | 2.6E−13 | SM |
| SM 42:3 | −15.8 | 3.9E−05 | SM |
| SM 44:1 | −23.7 | 1.1E−08 | SM |
| SM 44:3 | −16.6 | 1.3E−04 | SM |
| TAG(14:0/16:0/18:1) | −37.3 | 1.0E−04 | TAG II |
| TAG(14:0/16:0/18:2) | −36.5 | 2.3E−04 | TAG II |
| TAG(14:0/16:1/18:1) | −44.3 | 1.5E−04 | TAG II |
| TAG(14:0/16:1/18:2) | −50.9 | 2.4E−06 | TAG II |
| TAG(14:0/17:0/18:1) | −22.6 | 4.5E−02 | TAG II |
| TAG(14:0/18:0/18:1) | −44.4 | 1.7E−04 | TAG II |
| TAG(14:0/18:2/18:2) | −47.7 | 3.3E−06 | TAG II |
| TAG(14:1/16:0/18:1) | −35.4 | 4.8E−03 | TAG II |
| TAG(14:1/16:1/18:0) | −28.7 | 1.6E−02 | TAG II |
| TAG(14:1/18:0/18:2) | −25.3 | 1.4E−02 | TAG II |
| TAG(14:1/18:1/18:1) | −33.0 | 1.2E−04 | TAG II |
| TAG(16:1/16:1/16:1) | −43.7 | 5.2E−05 | TAG II |
| TAG(16:1/16:1/18:0) | −40.5 | 6.6E−05 | TAG II |
| TAG(16:1/18:1/18:2) | −13.9 | 3.8E−02 | TAG II |

Table 3 shows statistically significantly (p<0.05) increased or decreased small molecule biomarkers in ovarian cancer patients as compared to control group according to some embodiments of all aspects of the present disclosure. The results were derived from cohort I, except for acetoacetic acid, where its concentration was determined by more accurate method in cohort II.

TABLE 3
Increased (group A) (a) and decreased (group B) (b) small molecule biomarkers in
ovarian cancer patients according to some embodiments of the present disclosure.
Abbreviations are described in the detailed description of the present disclosure.

| | CHANGE (%) | P-VALUE | CLASS |
|---|---|---|---|
| Table 3a. Group A molecules | | | |
| Cysteine | 27.3 | 4.9E−02 | Amino acids I |
| Glutamic acid | 25.7 | 1.6E−03 | Amino acids I |
| Glutamine | 10.6 | 2.2E−02 | Amino acids I |
| 2-Aminobutyric acid | 99.7 | 2.1E−04 | Butyric acids |
| Cer(d16:1/18:0) | 24.6 | 1.3E−03 | Ceramides with FA 16:0, 18:0, 20:0, 22:0, 24:1 |
| Cer(d18:0/16:0) | 27.0 | 3.2E−03 | Ceramides with FA 16:0, 18:0, 20:0, 22:0, 24:1 |
| Cer(d18:0/18:0) | 49.7 | 5.7E−05 | Ceramides with FA 16:0, 18:0, 20:0, 22:0, 24:1 |
| Cer(d18:0/20:0) | 22.0 | 1.8E−02 | Ceramides with FA 16:0, 18:0, 20:0, 22:0, 24:1 |
| Cer(d18:0/24:1) | 16.4 | 1.5E−02 | Ceramides with FA 16:0, 18:0, 20:0, 22:0, 24:1 |
| Cer(d18:1/16:0) | 26.7 | 1.6E−05 | Ceramides with FA 16:0, 18:0, 20:0, 22:0, 24:1 |
| Cer(d18:1/18:0) | 71.4 | 7.2E−12 | Ceramides with FA 16:0, 18:0, 20:0, 22:0, 24:1 |
| Cer(d18:1/20:0) | 39.7 | 9.9E−08 | Ceramides with FA 16:0, 18:0, 20:0, 22:0, 24:1 |
| Cer(d18:1/22:0) | 11.9 | 2.1E−02 | Ceramides with FA 16:0, 18:0, 20:0, 22:0, 24:1 |
| Cer(d18:1/24:1) | 30.5 | 1.1E−06 | Ceramides with FA 16:0, 18:0, 20:0, 22:0, 24:1 |
| Cer(d18:2/16:0) | 15.2 | 1.2E−02 | Ceramides with FA 16:0, 18:0, 20:0, 22:0, 24:1 |
| Cer(d18:2/18:0) | 26.2 | 5.2E−04 | Ceramides with FA 16:0, 18:0, 20:0, 22:0, 24:1 |
| Cer(d18:2/24:1) | 13.5 | 1.5E−02 | Ceramides with FA 16:0, 18:0, 20:0, 22:0, 24:1 |
| Cer(d20:1/22:0) | 21.6 | 2.8E−03 | Ceramides with FA 16:0, 18:0, 20:0, 22:0, 24:1 |
| Cer(d20:1/24:1) | 43.9 | 2.1E−07 | Ceramides with FA 16:0, 18:0, 20:0, 22:0, 24:1 |
| Eicosenoic acid (C20:1) | 11.6 | 2.1E−02 | Fatty acids |
| Lauric acid (C12:0) | 14.9 | 4.1E−02 | Fatty acids |
| Linolenic acid (C18:3) | 37.3 | 2.3E−05 | Fatty acids |
| Myristic acid (C14:0) | 20.8 | 5.7E−03 | Fatty acids |
| Oleic acid (C18:1) | 16.7 | 6.0E−04 | Fatty acids |
| Palmitelaidic acid (C16:1) | 27.6 | 2.9E−04 | Fatty acids |
| LPC 20:1_sn2 | 20.6 | 3.8E−02 | LPL I |
| LPC 20:5_sn1 | 18.6 | 2.6E−02 | LPL I |
| LPC 22:6_sn1 | 20.5 | 3.7E−02 | LPL I |
| LPC O-18:1 | 35.9 | 1.9E−02 | LPL I |
| LPC P-18:1 | 29.2 | 4.4E−02 | LPL I |
| Alpha-ketoglutaric acid | 52.1 | 4.8E−02 | Other I |
| Ethanolamine | 49.2 | 3.1E−07 | Other I |
| Glycerol | 64.2 | 9.7E−14 | Other I |
| PE O-36:1 | 62.4 | 1.9E−05 | PE O I |
| PE O-36:2 | 93.3 | 6.5E−08 | PE O I |
| PE O-36:3 | 66.0 | 9.3E−05 | PE O I |
| Arabinose | 52.5 | 1.3E−05 | Sugars |
| Erythritol | 34.3 | 3.4E−05 | Sugars |
| Glucopyranose | 17.0 | 2.6E−02 | Sugars |
| Maltose | 814.4 | 5.0E−23 | Sugars |
| Mannonic acid | 103.7 | 6.0E−14 | Sugars |
| Melibiose | 392.4 | 6.6E−08 | Sugars |
| Myo-Inositol | 32.4 | 4.2E−07 | Sugars |
| Turanose | 434.5 | 2.4E−03 | Sugars |
| Xylitol | 30.1 | 5.0E−09 | Sugars |
| TAG(16:0/18:1/18:1) | 9.2 | 3.7E−02 | TAG I |
| TAG(18:0/18:2/18:2) | 18.1 | 1.2E−02 | TAG I |
| TAG(18:1/18:1/18:1) | 8.2 | 3.9E−02 | TAG I |
| TAG(18:1/18:1/20:4) | 28.4 | 6.7E−06 | TAG I |
| TAG(18:1/18:1/22:6) | 54.9 | 5.4E−08 | TAG I |
| TAG(18:2/18:2/18:2) | 118.1 | 3.3E−07 | TAG I |
| Table 3b. Group B molecules | | | |
| Methionine | −25.9 | 5.7E−17 | Amino acids II |
| Phenylalanine | −13.2 | 1.2E−03 | Amino acids II |
| Serine | −13.1 | 1.5E−03 | Amino acids II |
| Threonine | −36.3 | 1.4E−15 | Amino acids II |
| Tryptophan | −66.8 | 2.1E−21 | Amino acids II |
| Tyrosine | −24.9 | 1.5E−03 | Amino acids II |
| Valine | −8.2 | 1.2E−02 | Amino acids II |
| CE 14:0 | −22.3 | 4.1E−06 | CE |
| CE 14:1 | −22.0 | 4.7E−05 | CE |
| CE 15:0 | −16.8 | 2.0E−04 | CE |
| CE 16:0 | −12.5 | 1.4E−04 | CE |
| CE 16:2 | −21.1 | 2.0E−07 | CE |
| CE 17:0 | −16.5 | 1.2E−04 | CE |
| CE 17:1 | −10.1 | 2.1E−02 | CE |
| CE 18:0 | −26.7 | 4.9E−10 | CE |
| CE 18:1 | −12.7 | 1.9E−04 | CE |
| CE 18:2 | −21.9 | 7.2E−10 | CE |
| CE 19:1 | −0.9 | 4.6E−02 | CE |
| CE 20:2 | −23.3 | 2.6E−07 | CE |
| CE 20:3 | −21.3 | 5.8E−06 | CE |
| CE 20:4 | −7.4 | 5.0E−02 | CE |
| CE 22:3 | −15.8 | 3.9E−02 | CE |
| CE 22:4 | −14.9 | 1.6E−03 | CE |
| CE 22:5 | −35.3 | 2.0E−08 | CE |
| CE 24:6 | −22.7 | 1.2E−04 | CE |
| Cer(d16:1/23:0) | −24.9 | 5.7E−04 | Ceramides with FA 14:0, 23:0, 24:0, 26:0 |
| Cer(d16:1/24:0) | −29.3 | 6.1E−08 | Ceramides with FA 14:0, 23:0, 24:0, 26:0 |
| Cer(d16:1/26:0) | −17.1 | 4.0E−03 | Ceramides with FA 14:0, 23:0, 24:0, 26:0 |
| Cer(d18:0/23:0) | −17.4 | 3.8E−03 | Ceramides with FA 14:0, 23:0, 24:0, 26:0 |
| Cer(d18:0/24:0) | −14.8 | 2.5E−03 | Ceramides with FA 14:0, 23:0, 24:0, 26:0 |
| Cer(d18:1/14:0) | −10.3 | 4.0E−02 | Ceramides with FA 14:0, 23:0, 24:0, 26:0 |
| Cer(d18:1/24:0) | −11.9 | 8.6E−03 | Ceramides with FA 14:0, 23:0, 24:0, 26:0 |
| Cer(d18:2/23:0) | −18.5 | 5.6E−04 | Ceramides with FA 14:0, 23:0, 24:0, 26:0 |
| Cer(d18:2/24:0) | −22.4 | 1.1E−05 | Ceramides with FA 14:0, 23:0, 24:0, 26:0 |
| Cer(d18:2/26:0) | −14.2 | 4.0E−02 | Ceramides with FA 14:0, 23:0, 24:0, 26:0 |
| DAG(14:0/18:1) | −39.1 | 6.9E−04 | DAG |
| DAG(14:0/18:2) | −38.3 | 1.6E−03 | DAG |
| DAG(18:1/20:3) | −31.3 | 1.1E−02 | DAG |
| DAG(16:0/16:1) | −27.6 | 1.3E−02 | DAG |
| DAG(18:0/20:4) | −22.0 | 3.9E−02 | DAG |
| Gb3(d18:1/16:0) | −14.8 | 3.0E−04 | Gb3 |
| Gb3(d18:1/18:0) | −13.4 | 2.2E−02 | Gb3 |
| Gb3(d18:1/22:0) | −19.5 | 4.5E−04 | Gb3 |
| Gb3(d18:1/23:0) | −16.2 | 5.6E−03 | Gb3 |
| Gb3(d18:1/24:0) | −23.1 | 8.6E−05 | Gb3 |
| Glc/GalCer(d16:1/20:0) | −16.8 | 9.7E−04 | Glc/GalCer |
| Glc/GalCer(d16:1/22:0) | −23.4 | 1.5E−05 | Glc/GalCer |
| Glc/GalCer(d16:1/23:0) | −19.3 | 3.8E−04 | Glc/GalCer |
| Glc/GalCer(d16:1/24:0) | −23.1 | 5.0E−06 | Glc/GalCer |
| Glc/GalCer(d18:1/16:0) | −13.3 | 1.1E−02 | Glc/GalCer |
| Glc/GalCer(d18:1/18:0) | −16.1 | 7.5E−04 | Glc/GalCer |
| Glc/GalCer(d18:1/20:0) | −19.7 | 9.2E−05 | Glc/GalCer |
| Glc/GalCer(d18:1/22:0) | −25.9 | 2.6E−07 | Glc/GalCer |
| Glc/GalCer(d18:1/23:0) | −23.8 | 7.8E−07 | Glc/GalCer |
| Glc/GalCer(d18:1/24:0) | −24.2 | 4.7E−07 | Glc/GalCer |
| Glc/GalCer(d18:1/26:0) | −21.0 | 9.4E−05 | Glc/GalCer |
| Glc/GalCer(d18:2/20:0) | −23.3 | 1.7E−04 | Glc/GalCer |
| Glc/GalCer(d18:2/22:0) | −17.9 | 5.0E−04 | Glc/GalCer |
| Glc/GalCer(d18:2/23:0) | −21.6 | 5.2E−05 | Glc/GalCer |
| Glc/GalCer(d18:2/24:0) | −23.9 | 4.8E−08 | Glc/GalCer |
| LacCer(d16:1/16:0) | −21.1 | 5.3E−05 | LacCer |
| LacCer(d18:1/16:0) | −23.5 | 2.0E−08 | LacCer |
| LacCer(d18:1/22:0) | −27.7 | 7.6E−10 | LacCer |
| LacCer(d18:1/23:0) | −26.0 | 1.7E−07 | LacCer |
| LacCer(d18:1/24:0) | −23.2 | 1.5E−08 | LacCer |
| LacCer(d18:1/24:1) | −21.0 | 2.8E−06 | LacCer |
| LacCer(d18:2/16:0) | −25.2 | 5.5E−08 | LacCer |
| LacCer(d18:2/22:0) | −18.2 | 3.9E−02 | LacCer |
| LacCer(d18:2/24:0) | −19.7 | 4.0E−03 | LacCer |
| LacCer(d18:2/24:1) | −10.7 | 2.6E−02 | LacCer |
| LPC 20:0_sn1 | −16.6 | 3.4E−03 | LPL II |
| LPC 20:0_sn2 | −14.6 | 1.1E−02 | LPL II |
| LPC 20:2_sn1 | −5.7 | 3.7E−02 | LPL II |
| LPC 20:2_sn2 | −16.4 | 3.2E−05 | LPL II |
| LPC 20:3_sn1 | −16.1 | 2.3E−03 | LPL II |
| LPC 20:3_sn2 | −24.4 | 1.4E−06 | LPL II |
| LPC 22:0_sn1 | −28.7 | 6.3E−10 | LPL II |
| LPC 22:4_sn1 | −11.1 | 1.7E−02 | LPL II |
| LPC 22:4_sn2 | −19.6 | 4.9E−05 | LPL II |
| LPC 24:0_sn1 | −28.7 | 1.5E−12 | LPL II |
| LPC 24:0_sn2 | −28.9 | 2.3E−13 | LPL II |
| LPC O-20:0 | −15.5 | 7.1E−04 | LPL II |
| LPC O-22:0 | −25.3 | 4.8E−11 | LPL II |
| LPC O-22:1 | −17.5 | 1.9E−03 | LPL II |
| LPC O-24:0 | −26.6 | 2.9E−10 | LPL II |
| LPC O-24:1 | −17.8 | 8.2E−05 | LPL II |
| LPC O-24:2 | −33.0 | 1.2E−08 | LPL II |
| 2-oxo-3-methylpentanoic acid | −26.9 | 7.0E−09 | Other II |
| 2-Oxoisovaleric acid | −18.5 | 2.3E−02 | Other II |
| Cholesterol | −31.3 | 1.8E−06 | Other II |
| Glyceric acid | −28.1 | 6.5E−04 | Other II |
| Glycerol-3-phosphate | −21.4 | 8.2E−03 | Other II |
| Indole-3-acetic acid | −27.2 | 3.0E−03 | Other II |
| Ketoleucine | −45.2 | 7.0E−19 | Other II |
| Lactic acid | −29.0 | 3.2E−11 | Other II |
| Malic acid | −28.5 | 2.1E−06 | Other II |
| Pyruvic acid | −32.0 | 3.2E−02 | Other II |
| PC 28:0 | −59.0 | 1.5E−15 | PC |
| PC 30:0 | −43.4 | 4.0E−15 | PC |
| PC 30:1 | −52.1 | 2.4E−13 | PC |
| PC 30:2 | −61.4 | 2.1E−14 | PC |
| PC 31:0 | −26.0 | 1.6E−07 | PC |
| PC 31:1 | −36.9 | 1.9E−08 | PC |
| PC 32:0 | −15.5 | 4.3E−05 | PC |
| PC 32:1 | −29.0 | 1.9E−06 | PC |
| PC 32:2 | −52.5 | 4.0E−19 | PC |
| PC 32:3 | −58.5 | 4.2E−17 | PC |
| PC 33:0 | −17.1 | 2.0E−03 | PC |
| PC 33:1 | −25.2 | 5.2E−06 | PC |
| PC 33:2 | −35.6 | 6.9E−13 | PC |
| PC 33:3 | −42.0 | 4.2E−11 | PC |
| PC 34:0 | −11.9 | 3.6E−03 | PC |
| PC 34:3 | −37.9 | 9.7E−14 | PC |
| PC 34:4 | −52.1 | 5.6E−16 | PC |
| PC 34:5 | −41.3 | 1.4E−05 | PC |
| PC 35:0 | −21.3 | 5.5E−04 | PC |
| PC 35:1 | −15.5 | 4.0E−03 | PC |
| PC 35:2 | −26.3 | 2.6E−06 | PC |
| PC 35:3 | −39.8 | 3.1E−15 | PC |
| PC 35:4 | −25.8 | 1.5E−07 | PC |
| PC 36:0 | −21.7 | 1.3E−07 | PC |
| PC 36:2 | −35.1 | 2.0E−13 | PC |
| PC 36:3 | −36.0 | 2.2E−14 | PC |
| PC 36:5 | −32.1 | 1.4E−07 | PC |
| PC 36:6 | −46.4 | 2.0E−09 | PC |
| PC 36:7 | −27.4 | 2.1E−05 | PC |
| PC 37:1 | −20.5 | 4.6E−06 | PC |
| PC 37:2 | −36.3 | 6.0E−13 | PC |
| PC 37:3 | −30.4 | 7.4E−09 | PC |
| PC 37:6 | −20.3 | 8.9E−03 | PC |
| PC 38:0 | −29.1 | 5.0E−11 | PC |
| PC 38:1 | −9.8 | 2.8E−02 | PC |
| PC 38:2 | −27.9 | 3.7E−08 | PC |
| PC 38:3 | −30.6 | 1.5E−09 | PC |
| PC 38:4 | −24.1 | 6.4E−06 | PC |
| PC 38:5 | −15.7 | 6.6E−05 | PC |
| PC 38:6 | −35.0 | 2.6E−10 | PC |
| PC 38:7 | −30.9 | 7.8E−05 | PC |
| PC 39:0 | −18.7 | 9.9E−07 | PC |
| PC 39:4 | −19.5 | 5.6E−06 | PC |
| PC 39:5 | −12.0 | 3.5E−02 | PC |
| PC 40:1 | −22.9 | 3.6E−08 | PC |
| PC 40:3 | −15.8 | 1.6E−04 | PC |
| PC 40:4 | −29.9 | 9.0E−12 | PC |
| PC 40:5 | −14.8 | 1.4E−03 | PC |
| PC 40:8 | −33.4 | 6.8E−10 | PC |
| PC O-32:0 | −24.5 | 2.7E−08 | PC O/P |
| PC O-32:1 | −31.3 | 7.1E−10 | PC O/P |
| PC O-34:0 | −20.9 | 1.1E−06 | PC O/P |
| PC O-34:1 | −22.2 | 1.2E−08 | PC O/P |
| PC O-34:2 | −39.7 | 6.7E−17 | PC O/P |
| PC O-36:1 | −27.6 | 1.5E−07 | PC O/P |
| PC O-36:2 | −36.3 | 4.2E−14 | PC O/P |
| PC O-36:3 | −38.7 | 4.8E−16 | PC O/P |
| PC O-36:4 | −23.1 | 2.4E−09 | PC O/P |
| PC O-38:0 | −21.3 | 8.0E−09 | PC O/P |
| PC O-38:1 | −27.9 | 5.8E−12 | PC O/P |
| PC O-38:2 | −28.3 | 4.8E−09 | PC O/P |
| PC O-38:3 | −31.7 | 2.9E−11 | PC O/P |
| PC O-38:4 | −31.5 | 7.2E−12 | PC O/P |
| PC O-38:5 | −16.0 | 3.4E−05 | PC O/P |
| PC O-38:6 | −13.5 | 7.2E−03 | PC O/P |
| PC O-40:1 | −23.6 | 4.1E−06 | PC O/P |
| PC O-40:3 | −37.5 | 2.5E−10 | PC O/P |
| PC O-40:4 | −18.1 | 8.5E−04 | PC O/P |
| PC O-40:5 | −20.3 | 1.3E−04 | PC O/P |
| PC P-36:2 | −26.8 | 6.2E−08 | PC O/P |
| PC P-32:0 | −20.2 | 8.1E−07 | PC O/P |
| PC P-32:1 | −21.4 | 1.6E−05 | PC O/P |
| PC P-34:1 | −13.9 | 3.6E−04 | PC O/P |
| PC P-34:2 | −33.3 | 2.7E−13 | PC O/P |
| PC P-36:3 | −17.8 | 2.4E−06 | PC O/P |
| PC P-36:4 | −20.0 | 8.1E−07 | PC O/P |
| PC P-38:1 | −16.5 | 4.7E−04 | PC O/P |
| PC P-38:2 | −17.0 | 2.7E−03 | PC O/P |
| PC P-38:4 | −21.4 | 2.1E−05 | PC O/P |
| PC P-38:5 | −11.3 | 1.5E−03 | PC O/P |
| PC P-38:6 | −22.9 | 5.9E−04 | PC O/P |
| PC P-40:1 | −15.9 | 5.1E−04 | PC O/P |
| PC P-40:2 | −21.6 | 1.3E−05 | PC O/P |
| PC P-40:3 | −23.1 | 3.4E−07 | PC O/P |
| PC P-40:4 | −21.8 | 2.8E−07 | PC O/P |
| PC P-40:5 | −17.3 | 2.4E−05 | PC O/P |
| PC P-40:6 | −6.2 | 3.7E−02 | PC O/P |
| PE 32:1 | −39.1 | 2.3E−04 | PE |
| PE 34:2 | −39.2 | 1.0E−06 | PE |
| PE 34:3 | −49.7 | 8.1E−07 | PE |
| PE 36:3 | −53.8 | 2.0E−07 | PE |
| PE 36:4 | −30.7 | 2.9E−04 | PE |
| PE 36:5 | −26.5 | 2.3E−02 | PE |
| PE 38:1 | −37.4 | 7.9E−05 | PE |
| PE 38:2 | −29.4 | 2.0E−02 | PE |
| PE 38:3 | −40.4 | 6.8E−07 | PE |
| PE 38:4 | −47.6 | 7.3E−07 | PE |
| PE 38:5 | −33.5 | 5.1E−06 | PE |
| PE 40:4 | −34.8 | 1.2E−03 | PE |
| PE 40:5 | −21.8 | 8.3E−04 | PE |
| PE 40:7 | −42.4 | 1.3E−03 | PE |
| PE O-34:1 | −12.4 | 4.7E−02 | PE O II |
| PE O-36:4 | −49.1 | 4.9E−11 | PE O II |
| PE O-38:4 | −48.9 | 1.5E−12 | PE O II |
| PE O-38:5 | −38.5 | 1.3E−09 | PE O II |
| PE O-38:6 | −16.2 | 4.4E−02 | PE O II |
| PE P-34:2 | −20.6 | 1.4E−03 | PE P |
| PE P-36:2 | −41.7 | 7.0E−10 | PE P |
| PE P-36:4 | −31.6 | 1.0E−06 | PE P |
| PE P-38:4 | −36.4 | 7.4E−10 | PE P |
| PE P-38:6 | −31.2 | 1.4E−07 | PE P |
| PE P-40:6 | −20.6 | 1.6E−03 | PE P |
| PG 34:1 | −22.9 | 1.1E−02 | PG |
| PG 36:2 | −25.6 | 3.5E−04 | PG |
| PI 32:0 | −49.6 | 3.0E−04 | PI |
| PI 34:0 | −45.7 | 5.7E−04 | PI |
| PI 34:1 | −31.3 | 4.7E−04 | PI |
| PI 34:2 | −31.3 | 6.4E−06 | PI |
| PI 36:1 | −40.1 | 3.8E−07 | PI |
| PI 36:2 | −27.9 | 5.2E−06 | PI |
| PI 36:3 | −51.5 | 9.9E−10 | PI |
| PI 36:4 | −24.3 | 2.7E−03 | PI |
| PI 38:2 | −40.5 | 3.5E−07 | PI |
| PI 38:3 | −33.8 | 1.6E−09 | PI |
| PI 38:5 | −24.2 | 4.3E−05 | PI |
| PI 40:4 | −19.9 | 1.1E−03 | PI |
| S1P d16:1 | −20.0 | 1.4E−08 | S1P/SA1P |
| S1P d18:1 | −19.3 | 1.3E−07 | S1P/SA1P |
| S1P d18:2 | −29.3 | 1.2E−13 | S1P/SA1P |
| SA1P d18:0 | −24.8 | 5.3E−13 | S1P/SA1P |
| SM 30:2 | −35.8 | 2.2E−11 | SM |
| SM 31:0 | −12.2 | 4.2E−02 | SM |
| SM 31:1 | −24.7 | 1.2E−07 | SM |
| SM 31:2 | −12.3 | 7.5E−03 | SM |
| SM 32:0 | −24.2 | 8.6E−08 | SM |
| SM 32:2 | −28.0 | 3.3E−11 | SM |
| SM 33:1 | −11.6 | 4.7E−03 | SM |
| SM 33:2 | −9.2 | 4.4E−02 | SM |
| SM 34:0 | −15.1 | 5.0E−04 | SM |
| SM 34:1 | −13.2 | 4.6E−04 | SM |
| SM 34:2 | −15.8 | 2.3E−05 | SM |
| SM 35:0 | −25.3 | 3.2E−06 | SM |
| SM 35:1 | −10.3 | 9.7E−03 | SM |
| SM 35:2 | −9.3 | 9.1E−03 | SM |
| SM 36:3 | −14.6 | 2.7E−03 | SM |
| SM 37:0 | −10.1 | 1.6E−02 | SM |
| SM 37:1 | −14.8 | 1.9E−04 | SM |
| SM 37:2 | −34.8 | 1.4E−13 | SM |
| SM 38:0 | −22.5 | 2.0E−05 | SM |
| SM 38:1 | −20.4 | 1.0E−06 | SM |
| SM 38:3 | −20.2 | 8.5E−05 | SM |
| SM 39:0 | −18.0 | 7.1E−03 | SM |
| SM 39:2 | −21.6 | 1.5E−03 | SM |
| SM 40:0 | −27.0 | 5.9E−07 | SM |
| SM 40:3 | −19.8 | 2.6E−06 | SM |
| SM 41:0 | −32.5 | 1.3E−09 | SM |
| SM 41:2 | −17.0 | 7.1E−05 | SM |
| SM 42:0 | −30.9 | 3.8E−10 | SM |
| SM 42:3 | −15.8 | 3.9E−05 | SM |
| SM 44:1 | −23.7 | 1.1E−08 | SM |
| SM 44:3 | −16.6 | 1.3E−04 | SM |

Table 4 shows statistically significantly (p<0.05) increased or decreased lipid biomarkers in ovarian cancer patients as compared to control group according to some embodiments of all aspects of the present disclosure. The results were derived from cohort I.

TABLE 4
Increased (group A) (a) and decreased (group B) (b) lipid biomarkers in ovarian
cancer patients according to some embodiments of the present disclosure. Abbreviations
are described in the detailed description of the present disclosure.

| | CHANGE (%) | P-VALUE | CLASS |
|---|---|---|---|
| Table 4a. Group A molecules | | | |
| Cer(d16:1/18:0) | 24.6 | 1.3E−03 | Ceramides with FA 16:0, 18:0, 20:0, 22:0, 24:1 |
| Cer(d18:0/16:0) | 27.0 | 3.2E−03 | Ceramides with FA 16:0, 18:0, 20:0, 22:0, 24:1 |
| Cer(d18:0/18:0) | 49.7 | 5.7E−05 | Ceramides with FA 16:0, 18:0, 20:0, 22:0, 24:1 |
| Cer(d18:0/20:0) | 22.0 | 1.8E−02 | Ceramides with FA 16:0, 18:0, 20:0, 22:0, 24:1 |
| Cer(d18:0/24:1) | 16.4 | 1.5E−02 | Ceramides with FA 16:0, 18:0, 20:0, 22:0, 24:1 |
| Cer(d18:1/18:0) | 71.4 | 7.2E−12 | Ceramides with FA 16:0, 18:0, 20:0, 22:0, 24:1 |
| Cer(d18:1/20:0) | 39.7 | 9.9E−08 | Ceramides with FA 16:0, 18:0, 20:0, 22:0, 24:1 |
| Cer(d18:1/22:0) | 11.9 | 2.1E−02 | Ceramides with FA 16:0, 18:0, 20:0, 22:0, 24:1 |
| Cer(d18:1/24:1) | 30.5 | 1.1E−06 | Ceramides with FA 16:0, 18:0, 20:0, 22:0, 24:1 |
| Cer(d18:2/16:0) | 15.2 | 1.2E−02 | Ceramides with FA 16:0, 18:0, 20:0, 22:0, 24:1 |
| Cer(d18:2/18:0) | 26.2 | 5.2E−04 | Ceramides with FA 16:0, 18:0, 20:0, 22:0, 24:1 |
| Cer(d18:2/24:1) | 13.5 | 1.5E−02 | Ceramides with FA 16:0, 18:0, 20:0, 22:0, 24:1 |
| Cer(d20:1/22:0) | 21.6 | 2.8E−03 | Ceramides with FA 16:0, 18:0, 20:0, 22:0, 24:1 |
| Cer(d20:1/24:1) | 43.9 | 2.1E−07 | Ceramides with FA 16:0, 18:0, 20:0, 22:0, 24:1 |
| LPC 20:1_sn2 | 20.6 | 3.8E−02 | LPL I |
| LPC 20:5_sn1 | 18.6 | 2.6E−02 | LPL I |
| LPC O-18:1 | 35.9 | 1.9E−02 | LPL I |
| LPC P-18:1 | 29.2 | 4.4E−02 | LPL I |
| LPE 18:0_sn1 | 35.5 | 1.4E−03 | LPL I |
| LPE 18:0_sn2 | 33.5 | 2.6E−03 | LPL I |
| PE O-36:1 | 62.4 | 1.9E−05 | PE O I |
| PE O-36:2 | 93.3 | 6.5E−08 | PE O I |
| PE O-36:3 | 66.0 | 9.3E−05 | PE O I |
| Table 4b. Group B molecules | | | |
| CE 14:0 | −22.3 | 4.1E−06 | CE |
| CE 14:1 | −22.0 | 4.7E−05 | CE |
| CE 15:0 | −16.8 | 2.0E−04 | CE |
| CE 16:0 | −12.5 | 1.4E−04 | CE |
| CE 16:2 | −21.1 | 2.0E−07 | CE |
| CE 17:0 | −16.5 | 1.2E−04 | CE |
| CE 17:1 | −10.1 | 2.1E−02 | CE |
| CE 18:0 | −26.7 | 4.9E−10 | CE |
| CE 18:1 | −12.7 | 1.9E−04 | CE |
| CE 18:2 | −21.9 | 7.2E−10 | CE |
| CE 19:1 | −0.9 | 4.6E−02 | CE |
| CE 20:2 | −23.3 | 2.6E−07 | CE |
| CE 20:3 | −21.3 | 5.8E−06 | CE |
| CE 20:4 | −7.4 | 5.0E−02 | CE |
| CE 22:3 | −15.8 | 3.9E−02 | CE |
| CE 22:4 | −14.9 | 1.6E−03 | CE |
| CE 22:5 | −35.3 | 2.0E−08 | CE |
| CE 24:6 | −22.7 | 1.2E−04 | CE |
| Cer(d16:1/23:0) | −24.9 | 5.7E−04 | Ceramides with FA 14:0, 23:0, 24:0, 26:0 |
| Cer(d16:1/24:0) | −29.3 | 6.1E−08 | Ceramides with FA 14:0, 23:0, 24:0, 26:0 |
| Cer(d16:1/26:0) | −17.1 | 4.0E−03 | Ceramides with FA 14:0, 23:0, 24:0, 26:0 |
| Cer(d18:0/23:0) | −17.4 | 3.8E−03 | Ceramides with FA 14:0, 23:0, 24:0, 26:0 |
| Cer(d18:0/24:0) | −14.8 | 2.5E−03 | Ceramides with FA 14:0, 23:0, 24:0, 26:0 |
| Cer(d18:1/14:0) | −10.3 | 4.0E−02 | Ceramides with FA 14:0, 23:0, 24:0, 26:0 |
| Cer(d18:1/24:0) | −11.9 | 8.6E−03 | Ceramides with FA 14:0, 23:0, 24:0, 26:0 |
| Cer(d18:2/23:0) | −18.5 | 5.6E−04 | Ceramides with FA 14:0, 23:0, 24:0, 26:0 |
| Cer(d18:2/24:0) | −22.4 | 1.1E−05 | Ceramides with FA 14:0, 23:0, 24:0, 26:0 |
| Cer(d18:2/26:0) | −14.2 | 4.0E−02 | Ceramides with FA 14:0, 23:0, 24:0, 26:0 |
| DAG(14:0/18:1) | −39.1 | 6.9E−04 | DAG |
| DAG(14:0/18:2) | −38.3 | 1.6E−03 | DAG |
| DAG(18:1/20:3) | −31.3 | 1.1E−02 | DAG |
| DAG(16:0/16:1) | −27.6 | 1.3E−02 | DAG |
| DAG(18:0/20:4) | −22.0 | 3.9E−02 | DAG |
| Gb3(d18:1/16:0) | −14.8 | 3.0E−04 | Gb3 |
| Gb3(d18:1/18:0) | −13.4 | 2.2E−02 | Gb3 |
| Gb3(d18:1/22:0) | −19.5 | 4.5E−04 | Gb3 |
| Gb3(d18:1/23:0) | −16.2 | 5.6E−03 | Gb3 |
| Gb3(d18:1/24:0) | −23.1 | 8.6E−05 | Gb3 |
| Glc/GalCer(d16:1/20:0) | −16.8 | 9.7E−04 | Glc/GalCer |
| Glc/GalCer(d16:1/22:0) | −23.4 | 1.5E−05 | Glc/GalCer |
| Glc/GalCer(d16:1/23:0) | −19.3 | 3.8E−04 | Glc/GalCer |
| Glc/GalCer(d16:1/24:0) | −23.1 | 5.0E−06 | Glc/GalCer |
| Glc/GalCer(d18:1/16:0) | −13.3 | 1.1E−02 | Glc/GalCer |
| Glc/GalCer(d18:1/18:0) | −16.1 | 7.5E−04 | Glc/GalCer |
| Glc/GalCer(d18:1/20:0) | −19.7 | 9.2E−05 | Glc/GalCer |
| Glc/GalCer(d18:1/22:0) | −25.9 | 2.6E−07 | Glc/GalCer |
| Glc/GalCer(d18:1/23:0) | −23.8 | 7.8E−07 | Glc/GalCer |
| Glc/GalCer(d18:1/24:0) | −24.2 | 4.7E−07 | Glc/GalCer |
| Glc/GalCer(d18:1/26:0) | −21.0 | 9.4E−05 | Glc/GalCer |
| Glc/GalCer(d18:2/20:0) | −23.3 | 1.7E−04 | Glc/GalCer |
| Glc/GalCer(d18:2/22:0) | −17.9 | 5.0E−04 | Glc/GalCer |
| Glc/GalCer(d18:2/23:0) | −21.6 | 5.2E−05 | Glc/GalCer |
| Glc/GalCer(d18:2/24:0) | −23.9 | 4.8E−08 | Glc/GalCer |
| LacCer(d16:1/16:0) | −21.1 | 5.3E−05 | LacCer |
| LacCer(d18:1/22:0) | −27.7 | 7.6E−10 | LacCer |
| LacCer(d18:1/23:0) | −26.0 | 1.7E−07 | LacCer |
| LacCer(d18:1/24:0) | −23.2 | 1.5E−08 | LacCer |
| LacCer(d18:1/24:1) | −21.0 | 2.8E−06 | LacCer |
| LacCer(d18:2/16:0) | −25.2 | 5.5E−08 | LacCer |
| LacCer(d18:2/22:0) | −18.2 | 3.9E−02 | LacCer |
| LacCer(d18:2/24:0) | −19.7 | 4.0E−03 | LacCer |
| LacCer(d18:2/24:1) | −10.7 | 2.6E−02 | LacCer |
| LPC 20:0_sn1 | −16.6 | 3.4E−03 | LPL II |
| LPC 20:0_sn2 | −14.6 | 1.1E−02 | LPL II |
| LPC 20:2_sn1 | −5.7 | 3.7E−02 | LPL II |
| LPC 20:2_sn2 | −16.4 | 3.2E−05 | LPL II |
| LPC 22:0_sn1 | −28.7 | 6.3E−10 | LPL II |
| LPC 22:4_sn1 | −11.1 | 1.7E−02 | LPL II |
| LPC 22:4_sn2 | −19.6 | 4.9E−05 | LPL II |
| LPC 24:0_sn1 | −28.7 | 1.5E−12 | LPL II |
| LPC 24:0_sn2 | −28.9 | 2.3E−13 | LPL II |
| LPC O-20:0 | −15.5 | 7.1E−04 | LPL II |
| LPC O-22:0 | −25.3 | 4.8E−11 | LPL II |
| LPC O-22:1 | −17.5 | 1.9E−03 | LPL II |
| LPC O-24:0 | −26.6 | 2.9E−10 | LPL II |
| LPC O-24:1 | −17.8 | 8.2E−05 | LPL II |
| LPC O-24:2 | −33.0 | 1.2E−08 | LPL II |
| PC 28:0 | −59.0 | 1.5E−15 | PC |
| PC 30:0 | −43.4 | 4.0E−15 | PC |
| PC 30:1 | −52.1 | 2.4E−13 | PC |
| PC 30:2 | −61.4 | 2.1E−14 | PC |
| PC 31:0 | −26.0 | 1.6E−07 | PC |
| PC 31:1 | −36.9 | 1.9E−08 | PC |
| PC 32:0 | −15.5 | 4.3E−05 | PC |
| PC 32:1 | −29.0 | 1.9E−06 | PC |
| PC 32:2 | −52.5 | 4.0E−19 | PC |
| PC 32:3 | −58.5 | 4.2E−17 | PC |
| PC 33:0 | −17.1 | 2.0E−03 | PC |
| PC 33:1 | −25.2 | 5.2E−06 | PC |
| PC 33:2 | −35.6 | 6.9E−13 | PC |
| PC 33:3 | −42.0 | 4.2E−11 | PC |
| PC 34:0 | −11.9 | 3.6E−03 | PC |
| PC 34:3 | −37.9 | 9.7E−14 | PC |
| PC 34:5 | −41.3 | 1.4E−05 | PC |
| PC 35:0 | −21.3 | 5.5E−04 | PC |
| PC 35:1 | −15.5 | 4.0E−03 | PC |
| PC 35:2 | −26.3 | 2.6E−06 | PC |
| PC 35:3 | −39.8 | 3.1E−15 | PC |
| PC 35:4 | −25.8 | 1.5E−07 | PC |
| PC 36:0 | −21.7 | 1.3E−07 | PC |
| PC 36:5 | −32.1 | 1.4E−07 | PC |
| PC 36:6 | −46.4 | 2.0E−09 | PC |
| PC 36:7 | −27.4 | 2.1E−05 | PC |
| PC 37:1 | −20.5 | 4.6E−06 | PC |
| PC 37:2 | −36.3 | 6.0E−13 | PC |
| PC 37:3 | −30.4 | 7.4E−09 | PC |
| PC 37:6 | −20.3 | 8.9E−03 | PC |
| PC 38:0 | −29.1 | 5.0E−11 | PC |
| PC 38:1 | −9.8 | 2.8E−02 | PC |
| PC 38:7 | −30.9 | 7.8E−05 | PC |
| PC 39:0 | −18.7 | 9.9E−07 | PC |
| PC 39:4 | −19.5 | 5.6E−06 | PC |
| PC 39:5 | −12.0 | 3.5E−02 | PC |
| PC 40:1 | −22.9 | 3.6E−08 | PC |
| PC 40:3 | −15.8 | 1.6E−04 | PC |
| PC 40:4 | −29.9 | 9.0E−12 | PC |
| PC 40:8 | −33.4 | 6.8E−10 | PC |
| PC O-32:0 | −24.5 | 2.7E−08 | PC O/P |
| PC O-32:1 | −31.3 | 7.1E−10 | PC O/P |
| PC O-34:0 | −20.9 | 1.1E−06 | PC O/P |
| PC O-34:1 | −22.2 | 1.2E−08 | PC O/P |
| PC O-34:2 | −39.7 | 6.7E−17 | PC O/P |
| PC O-36:1 | −27.6 | 1.5E−07 | PC O/P |
| PC O-36:2 | −36.3 | 4.2E−14 | PC O/P |
| PC O-36:3 | −38.7 | 4.8E−16 | PC O/P |
| PC O-36:4 | −23.1 | 2.4E−09 | PC O/P |
| PC O-38:0 | −21.3 | 8.0E−09 | PC O/P |
| PC O-38:1 | −27.9 | 5.8E−12 | PC O/P |
| PC O-38:2 | −28.3 | 4.8E−09 | PC O/P |
| PC O-38:3 | −31.7 | 2.9E−11 | PC O/P |
| PC O-38:4 | −31.5 | 7.2E−12 | PC O/P |
| PC O-38:5 | −16.0 | 3.4E−05 | PC O/P |
| PC O-38:6 | −13.5 | 7.2E−03 | PC O/P |
| PC O-40:1 | −23.6 | 4.1E−06 | PC O/P |
| PC O-40:3 | −37.5 | 2.5E−10 | PC O/P |
| PC O-40:4 | −18.1 | 8.5E−04 | PC O/P |
| PC O-40:5 | −20.3 | 1.3E−04 | PC O/P |
| PC P-36:2 | −26.8 | 6.2E−08 | PC O/P |
| PC P-32:0 | −20.2 | 8.1E−07 | PC O/P |
| PC P-32:1 | −21.4 | 1.6E−05 | PC O/P |
| PC P-34:1 | −13.9 | 3.6E−04 | PC O/P |
| PC P-34:2 | −33.3 | 2.7E−13 | PC O/P |
| PC P-36:4 | −20.0 | 8.1E−07 | PC O/P |
| PC P-38:1 | −16.5 | 4.7E−04 | PC O/P |
| PC P-38:2 | −17.0 | 2.7E−03 | PC O/P |
| PC P-38:4 | −21.4 | 2.1E−05 | PC O/P |
| PC P-38:5 | −11.3 | 1.5E−03 | PC O/P |
| PC P-38:6 | −22.9 | 5.9E−04 | PC O/P |
| PC P-40:1 | −15.9 | 5.1E−04 | PC O/P |
| PC P-40:2 | −21.6 | 1.3E−05 | PC O/P |
| PC P-40:3 | −23.1 | 3.4E−07 | PC O/P |
| PC P-40:4 | −21.8 | 2.8E−07 | PC O/P |
| PC P-40:5 | −17.3 | 2.4E−05 | PC O/P |
| PE 32:1 | −39.1 | 2.3E−04 | PE |
| PE 34:2 | −39.2 | 1.0E−06 | PE |
| PE 34:3 | −49.7 | 8.1E−07 | PE |
| PE 36:2 | −37.9 | 1.5E−06 | PE |
| PE 36:3 | −53.8 | 2.0E−07 | PE |
| PE 36:4 | −30.7 | 2.9E−04 | PE |
| PE 36:5 | −26.5 | 2.3E−02 | PE |
| PE 38:1 | −37.4 | 7.9E−05 | PE |
| PE 38:2 | −29.4 | 2.0E−02 | PE |
| PE 38:3 | −40.4 | 6.8E−07 | PE |
| PE 38:5 | −33.5 | 5.1E−06 | PE |
| PE 40:4 | −34.8 | 1.2E−03 | PE |
| PE 40:5 | −21.8 | 8.3E−04 | PE |
| PE 40:7 | −42.4 | 1.3E−03 | PE |
| PE O-34:1 | −12.4 | 4.7E−02 | PE O II |
| PE O-36:4 | −49.1 | 4.9E−11 | PE O II |
| PE O-38:4 | −48.9 | 1.5E−12 | PE O II |
| PE O-38:5 | −38.5 | 1.3E−09 | PE O II |
| PE O-38:6 | −16.2 | 4.4E−02 | PE O II |
| PE P-34:2 | −20.6 | 1.4E−03 | PE P |
| PE P-36:2 | −41.7 | 7.0E−10 | PE P |
| PE P-36:4 | −31.6 | 1.0E−06 | PE P |
| PE P-38:4 | −36.4 | 7.4E−10 | PE P |
| PE P-38:6 | −31.2 | 1.4E−07 | PE P |
| PG 34:1 | −22.9 | 1.1E−02 | PG |
| PG 36:2 | −25.6 | 3.5E−04 | PG |
| PI 32:0 | −49.6 | 3.0E−04 | PI |
| PI 34:0 | −45.7 | 5.7E−04 | PI |
| PI 34:1 | −31.3 | 4.7E−04 | PI |
| PI 34:2 | −31.3 | 6.4E−06 | PI |
| PI 36:1 | −40.1 | 3.8E−07 | PI |
| PI 36:2 | −27.9 | 5.2E−06 | PI |
| PI 36:3 | −51.5 | 9.9E−10 | PI |
| PI 36:4 | −24.3 | 2.7E−03 | PI |
| PI 38:2 | −40.5 | 3.5E−07 | PI |
| PI 38:3 | −33.8 | 1.6E−09 | PI |
| PI 38:5 | −24.2 | 4.3E−05 | PI |
| PI 40:4 | −19.9 | 1.1E−03 | PI |
| SM 30:2 | −35.8 | 2.2E−11 | SM |
| SM 31:0 | −12.2 | 4.2E−02 | SM |
| SM 31:1 | −24.7 | 1.2E−07 | SM |
| SM 31:2 | −12.3 | 7.5E−03 | SM |
| SM 32:0 | −24.2 | 8.6E−08 | SM |
| SM 33:1 | −11.6 | 4.7E−03 | SM |
| SM 33:2 | −9.2 | 4.4E−02 | SM |
| SM 34:0 | −15.1 | 5.0E−04 | SM |
| SM 34:2 | −15.8 | 2.3E−05 | SM |
| SM 35:0 | −25.3 | 3.2E−06 | SM |
| SM 35:1 | −10.3 | 9.7E−03 | SM |
| SM 35:2 | −9.3 | 9.1E−03 | SM |
| SM 36:3 | −14.6 | 2.7E−03 | SM |
| SM 37:0 | −10.1 | 1.6E−02 | SM |
| SM 37:1 | −14.8 | 1.9E−04 | SM |
| SM 37:2 | −34.8 | 1.4E−13 | SM |
| SM 38:0 | −22.5 | 2.0E−05 | SM |
| SM 38:1 | −20.4 | 1.0E−06 | SM |
| SM 38:3 | −20.2 | 8.5E−05 | SM |
| SM 39:0 | −18.0 | 7.1E−03 | SM |
| SM 39:2 | −21.6 | 1.5E−03 | SM |
| SM 40:0 | −27.0 | 5.9E−07 | SM |
| SM 40:3 | −19.8 | 2.6E−06 | SM |
| SM 41:0 | −32.5 | 1.3E−09 | SM |
| SM 41:2 | −17.0 | 7.1E−05 | SM |
| SM 42:0 | −30.9 | 3.8E−10 | SM |
| SM 42:3 | −15.8 | 3.9E−05 | SM |
| SM 44:1 | −23.7 | 1.1E−08 | SM |
| SM 44:3 | −16.6 | 1.3E−04 | SM |

Tables 5-9 show examples of combinations of increasing and decreasing small molecule biomarkers from Group A and Group B, respectively, and are provided to illustrate various aspects of the present disclosure. They are not intended to limit the present disclosure, which is defined by the accompanying claims.

### Combination of Small Molecule Biomarkers Improves Diagnostic Performance

The embodiments of the present disclosure, i.e. combining increasing and decreasing small molecule biomarkers, improves diagnostic performance. This is illustrated in Table 5 by ratios of small molecule biomarkers. In Table 5 the results are shown for patients with malignant tumors vs. controls comparison in cohort I. When taking the ratio of two small molecule biomarkers, the performance measured by AUC as well as sum of sensitivity and specificity is improved when comparing the performance to single small molecule biomarker components of the ratios.

TABLE 5
Performance of small molecule ratios (a) and single small molecule
biomarkers (b) in ovarian cancer detection. Abbreviations are
described in the detailed description of the present disclosure.

| | AUC | SE + SP | SE | SP |
|---|---|---|---|---|
| Table 5a. Small molecule ratios | | | | |
| 2-Hydroxybutyric acid\|Ketoleucine | 0.895 | 1.66 | 0.92 | 0.73 |
| 3,4-Dihydroxybutyric acid\|Alanine | 0.910 | 1.68 | 0.97 | 0.71 |
| 3,4-Dihydroxybutyric acid\|PC 30:0 | 0.885 | 1.62 | 0.88 | 0.74 |
| 3,4-Dihydroxybutyric acid\|PC 32:2 | 0.886 | 1.62 | 0.74 | 0.89 |
| 3,4-Dihydroxybutyric acid\|PC 32:3 | 0.888 | 1.62 | 0.77 | 0.85 |
| 3,4-Dihydroxybutyric acid\|PC 34:4 | 0.870 | 1.57 | 0.73 | 0.84 |
| 3,4-Dihydroxybutyric acid\|PC 36:3 | 0.873 | 1.61 | 0.81 | 0.80 |
| 3,4-Dihydroxybutyric acid\|PC O-34:2 | 0.876 | 1.61 | 0.81 | 0.80 |
| 3,4-Dihydroxybutyric acid\|PC O-36:2 | 0.870 | 1.59 | 0.80 | 0.79 |
| 3,4-Dihydroxybutyric acid\|PC O-36:3 | 0.877 | 1.60 | 0.86 | 0.74 |
| 3,4-Dihydroxybutyric acid\|S1P d18:1 | 0.874 | 1.61 | 0.87 | 0.74 |
| 3,4-Dihydroxybutyric acid\|S1P d18:2 | 0.885 | 1.63 | 0.89 | 0.73 |
| 3-Hydroxybutyric acid\|Ketoleucine | 0.887 | 1.63 | 0.87 | 0.77 |
| 3-Hydroxybutyric acid\|PC O-34:2 | 0.875 | 1.60 | 0.82 | 0.79 |
| 3-Hydroxybutyric acid\|PC O-36:3 | 0.870 | 1.63 | 0.81 | 0.82 |
| 3-Hydroxybutyric acid\|PE O-38:4 | 0.871 | 1.58 | 0.86 | 0.71 |
| 3-Hydroxybutyric acid\|PE P-34:2 | 0.870 | 1.66 | 0.98 | 0.68 |
| 3-Hydroxybutyric acid\|SM 37:2 | 0.874 | 1.64 | 0.83 | 0.81 |
| Adipic acid\|Ketoleucine | 0.857 | 1.63 | 0.89 | 0.73 |
| Cer(d18:1/18:0)\|Alanine | 0.859 | 1.61 | 0.90 | 0.70 |
| Cer(d18:1/24:1)\|PC O-36:3 | 0.858 | 1.62 | 0.78 | 0.85 |
| LPE 18:0_sn1\|LPC 18:2_sn2 | 0.869 | 1.62 | 0.84 | 0.79 |
| Table 5b. Single small molecule biomarkers | | | | |
| 2-Hydroxybutyric acid | 0.724 | 1.38 | 0.82 | 0.56 |
| 3,4-Dihydroxybutyric acid | 0.853 | 1.63 | 0.73 | 0.90 |
| 3-Hydroxybutyric acid | 0.832 | 1.54 | 0.93 | 0.61 |
| Adipic acid | 0.712 | 1.39 | 0.68 | 0.70 |
| Alanine | 0.842 | 1.58 | 0.91 | 0.67 |
| Cer(d18:1/18:0) | 0.740 | 1.35 | 0.58 | 0.78 |
| Cer(d18:1/24:1) | 0.675 | 1.28 | 0.55 | 0.73 |
| Ketoleucine | 0.808 | 1.51 | 0.78 | 0.72 |
| LPC 18:2_sn2 | 0.755 | 1.43 | 0.72 | 0.70 |
| LPE 18:0_sn1 | 0.595 | 1.25 | 0.31 | 0.94 |
| PC 30:0 | 0.784 | 1.46 | 0.82 | 0.64 |
| PC 32:2 | 0.818 | 1.51 | 0.80 | 0.71 |
| PC 32:3 | 0.801 | 1.46 | 0.74 | 0.72 |
| PC 34:4 | 0.789 | 1.43 | 0.78 | 0.64 |
| PC 36:3 | 0.772 | 1.42 | 0.61 | 0.81 |
| PC O-34:2 | 0.795 | 1.45 | 0.59 | 0.87 |
| PC O-36:2 | 0.782 | 1.46 | 0.74 | 0.71 |
| PC O-36:3 | 0.801 | 1.52 | 0.70 | 0.82 |
| PE O-38:4 | 0.746 | 1.34 | 0.71 | 0.63 |
| PE P-34:2 | 0.658 | 1.28 | 0.50 | 0.78 |
| S1P d18:1 | 0.698 | 1.32 | 0.58 | 0.74 |
| S1P d18:2 | 0.788 | 1.50 | 0.88 | 0.62 |
| SM 37:2 | 0.777 | 1.45 | 0.66 | 0.79 |

### Combination of Small Molecule Biomarkers Improves Diagnostic Performance of Early Stage Ovarian Cancer Patients

Combination of increasing and decreasing small molecule biomarkers improves diagnostic performance also in the detection of early stage (stage I or II) ovarian cancer patients. This is illustrated in Table 6 by ratios of small molecule biomarkers. In Table 6 the results are shown for patients with stage I or II ovarian cancer vs. controls in cohort II. When taking the ratio of two small molecule biomarkers, the performance measured by AUC as well as sum of sensitivity and specificity is improved when comparing the performance to single small molecule biomarker components of the ratios.

TABLE 6
Performance of small molecule ratios (a) and single small molecule biomarkers
(b) in early-stage ovarian cancer detection. Abbreviations are described
in the detailed description of the present disclosure.

| | AUC | SE + SP | SE | SP |
|---|---|---|---|---|
| Table 6a. Small molecule ratios | | | | |
| Acetoacetic acid\|Gb3(d18:1/22:0) | 0.833 | 1.55 | 0.69 | 0.86 |
| Acetoacetic acid\|Glc/GalCer(d16:1/20:0) | 0.826 | 1.51 | 0.54 | 0.97 |
| Acetoacetic acid\|Glc/GalCer(d16:1/23:0) | 0.833 | 1.53 | 0.58 | 0.95 |
| Acetoacetic acid\|Glc/GalCer(d16:1/24:0) | 0.827 | 1.52 | 0.85 | 0.67 |
| Acetoacetic acid\|Ketoleucine | 0.828 | 1.53 | 0.77 | 0.76 |
| Acetoacetic acid\|LacCer(d18:1/23:0) | 0.844 | 1.57 | 0.77 | 0.80 |
| Acetoacetic acid\|PC 34:3 | 0.828 | 1.51 | 0.58 | 0.94 |
| Acetoacetic acid\|PC O-36:1 | 0.833 | 1.55 | 0.96 | 0.59 |
| Acetoacetic acid\|PC P-38:2 | 0.839 | 1.57 | 0.88 | 0.69 |
| Acetoacetic acid\|SM 31:2 | 0.832 | 1.55 | 0.65 | 0.89 |
| Cer(d18:0/18:0)\|LacCer(d18:1/23:0) | 0.755 | 1.46 | 0.71 | 0.75 |
| Cer(d18:1/24:1)\|LPC 20:2_sn2 | 0.785 | 1.49 | 0.81 | 0.69 |
| Cer(d18:1/24:1)\|PI 36:3 | 0.777 | 1.47 | 0.73 | 0.74 |
| Table 6b. Single small molecule biomarkers | | | | |
| Acetoacetic acid | 0.810 | 1.49 | 0.54 | 0.95 |
| Cer(d18:0/18:0) | 0.607 | 1.23 | 0.50 | 0.73 |
| Cer(d18:1/24:1) | 0.535 | 1.17 | 0.73 | 0.44 |
| Gb3(d18:1/22:0) | 0.668 | 1.29 | 0.46 | 0.83 |
| Glc/GalCer(d16:1/20:0) | 0.688 | 1.40 | 0.85 | 0.56 |
| Glc/GalCer(d16:1/23:0) | 0.640 | 1.25 | 0.62 | 0.63 |
| Glc/GalCer(d16:1/24:0) | 0.689 | 1.39 | 0.81 | 0.58 |
| Ketoleucine | 0.652 | 1.33 | 0.69 | 0.64 |
| LacCer(d18:1/23:0) | 0.713 | 1.46 | 0.73 | 0.73 |
| LPC 20:2_sn2 | 0.750 | 1.43 | 0.58 | 0.85 |
| PC 34:3 | 0.624 | 1.24 | 0.81 | 0.43 |
| PC O-36:1 | 0.673 | 1.44 | 0.73 | 0.71 |
| PC P-38:2 | 0.611 | 1.22 | 0.35 | 0.87 |
| PI 36:3 | 0.754 | 1.42 | 0.65 | 0.76 |
| SM 31:2 | 0.631 | 1.33 | 0.52 | 0.81 |

Combination of Small Molecule Biomarkers with CA-125 Improves Diagnostic Performance of Early Stage Ovarian Cancer Patients

Combination of increasing and decreasing small molecule biomarkers improves diagnostic performance also when combined together with protein biomarker CA-125. This is illustrated in Table 7 by ratios of small molecule biomarkers. In Table 7 the results are shown for patients with stage I or II ovarian cancer vs. controls in cohort II. When constructing a logistic regression model taking the ratio of two small molecule biomarkers and CA-125, the performance measured by AUC is improved when comparing the performance to a logistic regression model incorporating a single small molecule biomarker component and CA-125. Moreover, the AUC values are higher than those obtained for CA-125 alone.

TABLE 7
Performance of small molecule ratios (a) and single small
molecule biomarkers (b) in early-stage ovarian cancer
detection when combined with CA-125. (c) Performance
of CA-125 alone. Abbreviations are described in the
detailed description of the present disclosure.

| | AUC |
|---|---|
| Table 7a. Small molecule ratios | |
| 2-Hydroxybutyric acid\|SM 39:0 | 0.870 |
| 3-Hydroxybutyric acid\|SM 39:0 | 0.882 |
| Acetoacetic acid\|Cer(d16:1/26:0) | 0.914 |
| Acetoacetic acid\|Gb3(d18:1/22:0) | 0.921 |
| Acetoacetic acid\|Glc/GalCer(d16:1/22:0) | 0.913 |
| Acetoacetic acid\|Glc/GalCer(d16:1/23:0) | 0.914 |
| Acetoacetic acid\|LacCer(d16:1/16:0) | 0.915 |
| Acetoacetic acid\|PC 37:3 | 0.914 |
| Acetoacetic acid\|PC O-40:5 | 0.913 |
| Acetoacetic acid\|PC P-34:2 | 0.913 |
| Acetoacetic acid\|PC P-38:2 | 0.914 |
| Acetoacetic acid\|SM 31:2 | 0.920 |
| Acetoacetic acid\|SM 39:0 | 0.913 |
| Acetoacetic acid\|SM 39:1 | 0.913 |
| Cer(d18:1/24:1)\|LPC 14:0_sn1 | 0.874 |
| Cer(d18:1/24:1)\|PI 36:1 | 0.874 |
| Cer(d18:1/24:1)\|PI 36:3a | 0.882 |
| TAG(18:2/18:2/18:2)\|SM 39:0 | 0.880 |
| Table 7b. Single small molecule biomarkers | |
| 2-Hydroxybutyric acid | 0.807 |
| 3-Hydroxybutyric acid | 0.827 |
| Acetoacetic acid | 0.903 |
| Cer(d16:1/26:0) | 0.821 |
| Cer(d18:1/24:1) | 0.800 |
| Gb3(d18:1/22:0) | 0.839 |
| Glc/GalCer(d16:1/22:0) | 0.830 |
| Glc/GalCer(d16:1/23:0) | 0.816 |
| LacCer(d16:1/16:0) | 0.833 |
| LPC 14:0_sn1 | 0.853 |
| PC 37:3 | 0.869 |
| PC O-40:5 | 0.832 |
| PC P-34:2 | 0.835 |
| PC P-38:2 | 0.802 |
| PI 36:1 | 0.844 |
| PI 36:3a | 0.854 |
| SM 31:2 | 0.827 |
| SM 39:0 | 0.864 |
| SM 39:1 | 0.836 |
| TAG(18:2/18:2/18:2) | 0.816 |
| Table 7c. CA-125 value | |
| CA-125 | 0.806 |

### Combination of Small Molecule Biomarkers Improves Diagnostic Performance in Premenopausal Women

Combination of increasing and decreasing small molecule biomarkers improves diagnostic performance especially in premenopausal women suffering from ovarian cancer. This is illustrated in Table 8 by ratios of small molecule biomarkers. In Table 8 the results are shown for premenopausal patients with ovarian cancer vs. premenopausal controls in cohort II. When taking the ratio of two small molecule biomarkers, the performance measured by AUC as well as sum of sensitivity and specificity is improved when comparing the performance to single small molecule biomarker components of the ratios. The performance is improved also when compared to the clinically used protein biomarker CA-125.

TABLE 8
Performance of small molecule ratios (a), single small molecule biomarkers (b)
and CA-125 in detection of ovarian cancer in premenopausal women. Abbreviations
are described in the detailed description of the present disclosure.

| | AUC | SE + SP | SE | SP |
|---|---|---|---|---|
| Table 8a. Small molecule ratios | | | | |
| 2-Hydroxybutyric acid\|PC O-36:1 | 0.824 | 1.66 | 0.94 | 0.72 |
| Acetoacetic acid\|PC 30:2 | 0.825 | 1.51 | 0.65 | 0.87 |
| Cer(d18:1/24:1)\|LPC 14:0_sn1 | 0.832 | 1.53 | 0.76 | 0.77 |
| Cer(d20:1/24:1)\|LPC 14:0_sn1 | 0.830 | 1.56 | 0.94 | 0.62 |
| PE O-36:2\|PE P-36:2 | 0.839 | 1.64 | 0.94 | 0.69 |
| TAG(18:1/18:1/22:6)\|LPC 14:0_sn1 | 0.840 | 1.60 | 0.76 | 0.83 |
| TAG(18:1/18:1/22:6)\|LPC 14:0_sn2 | 0.839 | 1.58 | 0.76 | 0.82 |
| TAG(18:1/18:1/22:6)\|PC 36:6 | 0.855 | 1.59 | 0.82 | 0.77 |
| TAG(18:1/18:1/22:6)\|PC O-36:1 | 0.837 | 1.61 | 0.94 | 0.67 |
| TAG(18:1/18:1/22:6)\|TAG(14:0/18:2/18:2) | 0.861 | 1.72 | 0.88 | 0.83 |
| Table 8b. Single small molecule biomarkers | | | | |
| 2-Hydroxybutyric acid | 0.700 | 1.42 | 0.82 | 0.60 |
| Acetoacetic acid | 0.788 | 1.45 | 0.88 | 0.57 |
| Cer(d18:1/24:1) | 0.615 | 1.37 | 0.82 | 0.55 |
| Cer(d20:1/24:1) | 0.638 | 1.32 | 0.59 | 0.73 |
| LPC 14:0_sn1 | 0.818 | 1.57 | 0.88 | 0.68 |
| LPC 14:0_sn2 | 0.790 | 1.51 | 0.71 | 0.80 |
| PC 30:2 | 0.802 | 1.51 | 0.82 | 0.68 |
| PC 36:6 | 0.748 | 1.51 | 0.65 | 0.87 |
| PC O-36:1 | 0.790 | 1.62 | 0.88 | 0.73 |
| PE O-36:2 | 0.520 | 1.16 | 0.65 | 0.52 |
| PE P-36:2 | 0.810 | 1.56 | 0.88 | 0.68 |
| TAG(14:0/18:2/18:2) | 0.744 | 1.50 | 0.76 | 0.73 |
| TAG(18:1/18:1/22:6) | 0.680 | 1.34 | 0.82 | 0.52 |
| Table 8c. Result for CA-125 | | | | |
| CA125 | 0.777 | 1.51 | 0.94 | 0.57 |

Combination of Small Molecule Biomarkers Improves Prediction of Overall Survival in Patients with Ovarian Cancer

Combination of increasing and decreasing small molecule biomarkers improves the prediction of overall survival in patients suffering from ovarian cancer. This is illustrated in Table 9 by ratios of small molecule biomarkers. When taking the ratio of two small molecule biomarkers, the performance measured by hazard ratios and p-values of the cox regression models are improved when comparing the performance to single small molecule biomarker components of the ratios. The performance is improved also when compared to the clinically used protein biomarker CA-125.

TABLE 9
Performance of small molecule ratios (a), single small molecule
biomarkers (b) and CA-125 in predicting the overall survival
of ovarian cancer patients. Abbreviations are described in
the detailed description of the present disclosure.

| | HR (95% CI) | P-value |
|---|---|---|
| Table 9a. Small molecule ratios | | |
| 3,4-Dihydroxybutyric acid\|PC | 1.49 (1.19, 1.87) | 5.0E−04 |
| 37:6 | | |
| Adipic acid\|PC O-38:2 | 1.57 (1.26, 1.96) | 5.0E−05 |
| Cer(d18:1/16:0)\|PC O-38:2 | 1.81 (1.42, 2.32) | 2.4E−06 |
| Cer(d18:1/16:0)\|SM 41:1 | 1.72 (1.36, 2.18) | 4.8E−06 |
| Cer(d20:1/24:1)\|CE 20:3 | 1.80 (1.40, 2.31) | 3.8E−06 |
| Cer(d20:1/24:1)\|PC 37:3 | 1.90 (1.45, 2.49) | 3.0E−06 |
| Cer(d20:1/24:1)\|PC 37:6 | 1.84 (1.44, 2.36) | 1.4E−06 |
| Cer(d20:1/24:1)\|PC 40:8 | 2.05 (1.51, 2.77) | 3.4E−06 |
| Cer(d20:1/24:1)\|PC O-38:1 | 1.81 (1.41, 2.33) | 3.1E−06 |
| Cer(d20:1/24:1)\|PC O-38:2 | 1.86 (1.45, 2.40) | 1.4E−06 |
| Cer(d20:1/24:1)\|SM 41:1 | 1.82 (1.42, 2.34) | 2.7E−06 |
| TAG(18:1/18:1/20:4)\|PC 37:3 | 1.51 (1.19, 1.91) | 5.7E−04 |
| Table 9b. Single small molecule | | |
| biomarkers | | |
| 3,4-Dihydroxybutyric acid | 1.38 (1.10, 1.73) | 6.0E−03 |
| Adipic acid | 1.37 (1.09, 1.70) | 5.8E−03 |
| CE 20:3 | 0.75 (0.61, 0.93) | 7.7E−03 |
| Cer(d18:1/16:0) | 1.42 (1.12, 1.80) | 3.4E−03 |
| Cer(d20:1/24:1) | 1.53 (1.20, 1.94) | 5.3E−04 |
| PC 37:3 | 0.74 (0.60, 0.92) | 5.6E−03 |
| PC 37:6 | 0.72 (0.57, 0.90) | 3.4E−03 |
| PC 40:8 | 0.75 (0.59, 0.94) | 1.5E−02 |
| PC O-38:1 | 0.72 (0.58, 0.88) | 1.9E−03 |
| PC O-38:2 | 0.65 (0.52, 0.82) | 2.1E−04 |
| SM 41:1 | 0.72 (0.59, 0.87) | 9.5E−04 |
| TAG(18:1/18:1/20:4) | 1.24 (0.98, 1.55) | 6.8E−02 |
| Table 9c. Clinically used CA125 | | |
| marker | | |
| CA125 | 1.15 (0.85, 1.57) | 3.6E−01 |

Combination of Small Molecule Biomarkers with CA-125 Improves Diagnostic Performance of Early Stage Ovarian Cancer Patients

Combination of cancer antigen 125 (CA-125) and decreasing small molecule biomarkers (group B) improves diagnostic performance also in the detection of early stage (stage I or II) ovarian cancer patients. This is illustrated in Table 10 by ratios of CA-125 and small molecule biomarkers. In Table 10 the results are shown for patients with stage I or II ovarian cancer vs. controls in cohort II. When taking the ratio of CA-125 and a small molecule biomarker, the performance measured by AUC as well as sum of sensitivity and specificity is improved when comparing the performance to CA-125 or single small molecule biomarker alone.

TABLE 10
(a) Performance of ratios of CA-125 and small molecule
biomarkers from group B in early-stage ovarian cancer
detection, (b) Performance of CA-125 and single small
molecule biomarkers alone. Abbreviations are described
in the detailed description of the present disclosure.

| | AUC | SE + SP | SE | SP |
|---|---|---|---|---|
| Table 10a. CA-125/small | | | | |
| molecule ratios | | | | |
| CA-125\|LPC 14:0_sn2 | 0.858 | 1.62 | 0.88 | 0.73 |
| CA-125\|PI 36:3a | 0.855 | 1.61 | 0.85 | 0.77 |
| CA-125\|PC 37:3 | 0.854 | 1.59 | 0.88 | 0.70 |
| CA-125\|LPC 14:0_sn1 | 0.853 | 1.60 | 0.85 | 0.75 |
| CA-125\|LPC 20:3_sn1 | 0.847 | 1.61 | 0.88 | 0.72 |
| CA-125\|PI 36:3b | 0.845 | 1.61 | 0.85 | 0.77 |
| CA-125\|PC 34:4 | 0.845 | 1.59 | 0.92 | 0.67 |
| CA-125\|PC 40:4 | 0.843 | 1.64 | 0.92 | 0.71 |
| CA-125\|SM 39:0 | 0.842 | 1.61 | 0.87 | 0.74 |
| CA-125\|LPC 20:2_sn2 | 0.842 | 1.60 | 0.88 | 0.72 |
| CA-125\|PI 36:1 | 0.842 | 1.60 | 0.88 | 0.71 |
| CA-125\|S1P d18:2 | 0.838 | 1.62 | 0.92 | 0.70 |
| CA-125\|SM 39:1 | 0.836 | 1.64 | 0.88 | 0.76 |
| CA-125\|PI 38:5b | 0.836 | 1.62 | 0.92 | 0.69 |
| CA-125\|PC 36:3b + 1 | 0.834 | 1.65 | 0.81 | 0.84 |
| CA-125\|PC P-34:2 + 1 | 0.830 | 1.64 | 0.88 | 0.76 |
| CA-125\|LPC 24:0_sn1 | 0.830 | 1.62 | 0.88 | 0.74 |
| CA-125\|SM 31:2 | 0.826 | 1.63 | 0.78 | 0.85 |
| CA-125\|SM 31:1 | 0.824 | 1.63 | 0.85 | 0.79 |
| CA-125\|SM 32:1 | 0.821 | 1.63 | 0.85 | 0.79 |
| CA-125\|Gb3(d18:1/23:0) | 0.819 | 1.62 | 0.91 | 0.71 |
| Table 10b. CA-125 or single | | | | |
| small molecule biomarkers | | | | |
| CA-125 | 0.806 | 1.55 | 0.92 | 0.63 |
| LPC 14:0_sn2 | 0.713 | 1.34 | 0.85 | 0.50 |
| PI 36:3a | 0.754 | 1.42 | 0.65 | 0.76 |
| PC 37:3 | 0.737 | 1.47 | 0.73 | 0.74 |
| LPC 14:0_sn1 | 0.731 | 1.43 | 0.85 | 0.59 |
| LPC 20:3_sn1 | 0.722 | 1.39 | 0.65 | 0.73 |
| PI 36:3b | 0.691 | 1.36 | 0.50 | 0.86 |
| PC 34:4 | 0.651 | 1.27 | 0.85 | 0.42 |
| PC 40:4 | 0.715 | 1.39 | 0.73 | 0.66 |
| SM 39:0 | 0.555 | 1.20 | 0.83 | 0.38 |
| LPC 20:2_sn2 | 0.750 | 1.43 | 0.58 | 0.85 |
| PI 36:1 | 0.715 | 1.38 | 0.73 | 0.65 |
| S1P d18:2 | 0.695 | 1.35 | 0.92 | 0.43 |
| SM 39:1 | 0.686 | 1.43 | 0.69 | 0.73 |
| PI 38:5b | 0.645 | 1.27 | 0.42 | 0.84 |
| PC 36:3b + 1 | 0.658 | 1.30 | 0.81 | 0.50 |
| PC P-34:2 + 1 | 0.742 | 1.41 | 0.88 | 0.52 |
| LPC 20:4_sn1 | 0.685 | 1.34 | 1.00 | 0.34 |
| SM 31:2 | 0.631 | 1.33 | 0.52 | 0.81 |
| SM 31:1 | 0.642 | 1.35 | 0.50 | 0.85 |
| SM 32:1 | 0.662 | 1.29 | 0.73 | 0.56 |
| Gb3(d18:1/23:0) | 0.600 | 1.29 | 0.59 | 0.70 |

Combination of Small Molecule Biomarkers with CA-125 Improves Diagnostic Performance in Premenopausal Women

Combination of cancer antigen 125 (CA-125) and decreasing small molecule biomarkers (group B) improves diagnostic performance especially in premenopausal women suffering from ovarian cancer. This is illustrated in Table 11 by ratios of CA-125 and small molecule biomarkers. In Table 11 the results are shown for premenopausal patients with ovarian cancer vs. premenopausal controls in cohort II. When taking the ratio of CA-125 and a small molecule biomarker, the performance measured by AUC as well as sum of sensitivity and specificity is improved when comparing the performance to CA-125 or a single small molecule biomarker alone.

TABLE 11
(a) Performance of ratios of CA-125 and small molecule biomarkers
from group B in detection of ovarian cancer in premenopausal
patients. (b) Performance of CA-125 and single small molecule
biomarkers alone. Abbreviations are described in the detailed
description of the present disclosure.

| | AUC | SE + SP | SE | SP |
|---|---|---|---|---|
| Table 11a. CA-125/small | | | | |
| molecule ratios | | | | |
| CA-125\|PC 30:2 | 0.875 | 1.65 | 1.00 | 0.65 |
| CA-125\|PC 28:0 | 0.864 | 1.59 | 0.82 | 0.77 |
| CA-125\|PC 32:2 | 0.863 | 1.60 | 1.00 | 0.60 |
| CA-125\|PC 32:3 | 0.855 | 1.62 | 0.88 | 0.73 |
| CA-125\|LPC 20:0_sn1 | 0.851 | 1.62 | 0.82 | 0.80 |
| CA-125\|TAG(14:0/18:2/18:2) | 0.850 | 1.58 | 0.88 | 0.70 |
| CA-125\|PC 36:2 + 2 | 0.846 | 1.61 | 0.94 | 0.67 |
| CA-125\|PC 35:3b | 0.843 | 1.55 | 0.88 | 0.67 |
| CA-125\|PC 36:3a + 1 | 0.842 | 1.62 | 0.94 | 0.68 |
| CA-125\|LPC 22:0_sn1 | 0.841 | 1.59 | 0.82 | 0.76 |
| CA-125\|PC 37:2 | 0.841 | 1.54 | 0.82 | 0.72 |
| CA-125\|TAG(14:0/16:1/18:2) | 0.839 | 1.53 | 0.76 | 0.77 |
| CA-125\|SM 31:1 | 0.828 | 1.57 | 0.88 | 0.68 |
| CA-125\|SM 37:2 | 0.828 | 1.59 | 0.94 | 0.65 |
| CA-125\|DAG(14:0/18:2) | 0.827 | 1.54 | 0.86 | 0.68 |
| CA-125\|SM 39:1 | 0.847 | 1.61 | 0.94 | 0.67 |
| CA-125\|LPC 14:0_sn1 | 0.862 | 1.60 | 0.88 | 0.72 |
| CA-125\|SM 30:2 | 0.819 | 1.60 | 0.76 | 0.83 |
| CA-125\|LPC 20:3_sn2 | 0.837 | 1.59 | 0.94 | 0.65 |
| Table 11b. CA-125 or single | | | | |
| small molecule biomarkers | | | | |
| CA-125 | 0.777 | 1.51 | 0.94 | 0.57 |
| PC 30:2 | 0.802 | 1.51 | 0.82 | 0.68 |
| PC 28:0 | 0.779 | 1.46 | 0.71 | 0.75 |
| PC 32:2 | 0.782 | 1.52 | 0.71 | 0.82 |
| PC 32:3 | 0.756 | 1.51 | 0.76 | 0.75 |
| LPC 20:0_sn1 | 0.795 | 1.56 | 0.82 | 0.73 |
| TAG(14:0/18:2/18:2) | 0.744 | 1.50 | 0.76 | 0.73 |
| PC 36:2 + 2 | 0.784 | 1.52 | 0.71 | 0.82 |
| PC 35:3b | 0.768 | 1.52 | 0.88 | 0.63 |
| PC 36:3a +1 | 0.805 | 1.54 | 0.82 | 0.72 |
| LPC 22:0_sn1 | 0.812 | 1.52 | 0.82 | 0.69 |
| PC 37:2 | 0.797 | 1.51 | 0.71 | 0.80 |
| TAG(14:0/16:1/18:2) | 0.726 | 1.47 | 0.94 | 0.53 |
| SM 31:1 | 0.750 | 1.51 | 0.82 | 0.68 |
| SM 37:2 | 0.743 | 1.46 | 0.71 | 0.75 |
| DAG(14:0/18:2) | 0.707 | 1.39 | 0.71 | 0.68 |
| SM 39:1 | 0.762 | 1.57 | 0.88 | 0.68 |
| LPC 14:0_sn1 | 0.818 | 1.57 | 0.88 | 0.68 |
| SM 30:2 | 0.683 | 1.37 | 0.88 | 0.48 |
| LPC 20:3_sn2 | 0.768 | 1.50 | 0.76 | 0.73 |

1. An in vitro screening method for assessing whether a subject is at risk to develop or is suffering from ovarian cancer comprising:
(a) assaying a sample from said subject to determine a concentration of at least one small molecule biomarker from Group A;
(b) assaying a sample from said subject to determine a concentration of at least one small molecule biomarker from Group B; and
(c) determining that the subject is suffering from or is at an increased risk of developing ovarian cancer, if said sample contains an increased concentration of the at least one small molecule biomarker from Group A and a decreased concentration of the at least one small molecule biomarker from Group B, when compared to a control.

2. The method of claim 1, wherein the method further comprises determining from said sample a concentration of at least one protein biomarker, wherein an increase or decrease in the concentration of said protein biomarker(s), when compared to a control, is indicative of said subject suffering from or having an increased risk of developing ovarian cancer.

3.-4. (canceled)

5. A method of assessing whether a subject has a decreased or poor survival prognosis for ovarian cancer comprising:
(a) assaying a sample from said subject to determine a concentration of at least one small molecule biomarker from Group A;
(b) assaying a sample from said subject to determine a concentration of at least one small molecule biomarker from Group B; and
(c) determining that the subject has a decreased or poor survival prognosis, if said sample contains an increased concentration of the at least one small molecule biomarker from Group A and a decreased concentration of the at least one small molecule biomarker from Group B, when compared to a control.

6. The method of claim 5, wherein the method further comprises determining from said sample a concentration of at least one protein biomarker, wherein an increase or decrease in concentration of said protein biomarker(s), when compared to a control, is indicative of said subject having a decreased or poor survival prognosis.

7.-8. (canceled)

9. An in vitro method of evaluating the effectiveness of ovarian cancer therapy in a subject comprising:
(a) assaying a sample from said subject to determine a concentration of at least one small molecule biomarker from Group A;
(b) assaying a sample from said subject to determine a concentration of at least one small molecule biomarker from Group B; and
(c) determining that the therapy is effective, if said sample contains a decreased concentration of the at least one small molecule biomarker from Group A and an increased concentration of the at least one small molecule biomarker from Group B, when compared to a control.

10. The method of claim 9, wherein the method further comprises determining from said sample a concentration of at least one protein biomarker, wherein an increase or decrease in concentration of said protein biomarker(s), when compared to a control, is indicative of the effectiveness of the therapy.

11.-46. (canceled)

47. The method of claim 1, wherein the method comprises determining the concentration of at least 1, at least 2, at least 3, at least 4, at least 5, at least 6, at least 7, at least 8, at least 9, at least 10 or more small molecule biomarkers from Group A and at least 1, at least 2, at least 3, at least 4, at least 5, at least 6, at least 7, at least 8, at least 9, at least 10 or more small molecule biomarkers from Group B.

48. The method of claim 1, wherein the small molecule biomarker concentration is determined by using mass spectrometry, nuclear magnetic resonance spectroscopy, fluorescence spectroscopy or dual polarization interferometry, an immunoassay, enzymatic assay, colorimetric assay, fluorometric assay, a rapid test and/or with a binding moiety capable of specifically binding the biomarker.

49. (canceled)

50. The method of claim 1, wherein the sample is a serum sample or a plasma sample.

51. The method of claim 1, further comprising a step of spiking the sample with at least one isotope-labelled small molecule biomarker from Group A and/or at least one isotope-labelled small molecule biomarker from Group B.

52. The method of claim 51, wherein the at least one isotope-labelled small molecule biomarker from Group A and/or at least one isotope-labelled small molecule biomarker from Group B is/are deuterium-labelled.

53. The method of claim 1, wherein the ovarian cancer is early stage ovarian cancer.

54. The method of claim 1, wherein the method further comprises after the determining step (c), (d) diagnosing the subject as suffering from or having an increased risk of developing ovarian cancer from the results in step (c), and (e) administering a treatment to the subject diagnosed in step (d).

55.-57. (canceled)

58. The method of claim 5, wherein the method further comprises after the determining step (c), (d) administering a treatment to the subject.

59.-61. (canceled)

62. The method of claim 9, wherein the method further comprises (d) determining that the therapy is not effective in the subject from the results in step (c), and (e) escalating the therapy of the subject.

63. (canceled)

64. The method of claim 54, wherein the treatment is a surgery, chemotherapy, radiation therapy, hormonal therapy, anti-angiogenic therapy, therapies targeting homologous recombination deficiency, antibody therapy, other targeted therapy utilizing ovarian cancer specific signalling pathways, or any therapeutic treatment or operation typically given to a subject having ovarian cancer; or the treatment comprises administering a pharmaceutical agent affecting lipid metabolism.

65. (canceled)

66. The method of claim 54, wherein the method further comprises requesting a test from a laboratory which provides the results of an assay useful for determining the concentration of the at least one small molecule biomarker from Group A and the at least one small molecule biomarker from Group B, and administering to the subject a treatment if the subject has an increased concentration of the at least one small molecule biomarker from Group A and a decreased concentration of the at least one small molecule biomarker from Group B, as compared to the control.

67.-69. (canceled)

70. The method of claim 2, wherein the at least one protein biomarker is CA-125 and/or HE4, and wherein an increase in the concentration of said protein biomarker(s), when compared to a control, is indicative of said subject suffering from or having an increased risk of developing ovarian cancer.

71.-73. (canceled)

74. The method of claim 1, wherein the at least one small molecule biomarker from Group A and/or the at least one small molecule biomarker from Group B is(are) selected from the small molecule biomarkers listed in Table 2, Table 3 and/or Table 4.

75.-76. (canceled)

77. The method of claim 1, wherein the subject is a premenopausal subject.
